(19) United States
(12) Patent Application Publication
(10) Pub. No.: US 2014/0093900 A1
(43) Pub. Date: Apr. 3, 2014

(54) STEROID PROFILE IN OVARIAN FOLLICULAR FLUID FOR DIAGNOSIS, PROGNOSIS AND DETERMINING STRATEGIES FOR TREATMENT

(71) Applicants: Mark M. Kushnir, Salt Lake City, ND (US); Jonas Bergquist, Uppsala (SE); Tord Naessen, Uppsala (SE)

(72) Inventors: Mark M. Kushnir, Salt Lake City, ND (US); Jonas Bergquist, Uppsala (SE); Tord Naessen, Uppsala (SE)

(21) Appl. No.: 14/099,037

(22) Filed: Dec. 6, 2013

Related U.S. Application Data
(63) Continuation of application No. 12/936,503, filed on Jan. 3, 2011, now abandoned.

(51) Int. Cl. G01N 30/72; H01J 49/26; C12Q 1/26

(52) U.S. Cl. CPC G01N 30/72; C12Q 1/26; H01J 49/26
USPC 435/25; 250/282; 435/29

(57) ABSTRACT

Concentrations of endogenous steroids in ovarian follicular fluid are used to develop steroid profiles which provide means for the diagnosis and prognosis of endocrine-related conditions and for identifying and developing appropriate treatments for related conditions, including the identification and development of suitable protocols for in vitro fertilization (IVF), treatment and predictive strategies for successful IVF outcomes and selected uses of oocytes for IVF or embryonic stem cell procedures.

### TECHNICAL FIELD

This invention relates to the field of biotechnology, and more particularly to the use of steroid profiles derived from analysis of ovarian follicular fluid as biomarkers for diagnosis of and/or prognosis for a subject's condition, and for predicting the viability of oocytes for selected biological procedures, especially in vitro fertilization.

### BACKGROUND

The references discussed herein are provided solely for the purpose of describing the field relating to the invention. Nothing herein is to be construed as an admission that the inventors are not entitled to antedate a disclosure by virtue of prior invention.

In women of fertile age, the ovarian follicles are the main source for the synthesis of estrogens; ovarian follicles also contribute to circulating androgens with the adrenal cortex serving as another source of circulating androgens. Follicular steroids are secreted by granulose and theca cells under the control of gonadotropins, and this hormonal microenvironment affects development of the follicles and oocyte viability (1). A higher concentration of estradiol (E2) in follicular fluid (FF) is associated with healthy mature follicles containing oocytes that are capable of meiosis, while higher concentrations of androgens are indicative of atretic changes (1, 2). With the introduction of in vitro fertilization (IVF) a number of studies have focused on analyzing FF from women receiving ovarian stimulation. The majority of these studies were undertaken to obtain prognostic parameters for the likelihood of a successful implantation (3). However, relatively few publications have focused on the steroid hormones present in FF of regularly menstruating (RM) women and the relationship of the steroids to follicular development (4).

Polycystic ovary syndrome (PCOS) is one of the most common reproductive endocrine disorders, affecting about 5-8% of reproductive-age women, and is characterized by hyperandrogenism and anovulatory infertility (5). In PCOS patients, the chronic absence of ovulations results in accumulation in the ovaries of large number of atretic follicles, which produce the excess of androgens that leads to hyperandrogenism. In addition to reproductive abnormalities and hyperandrogenism, symptoms characteristic of PCOS may also include low FSH levels combined with high LH levels, obesity, hyperinsulinemia, type II diabetes, dyslipidemia, menstrual disorders, anovulation, hyperandrogenism, hirsutism, acne, a higher incidence of cardiovascular disease, and increased risk of endometrial and breast cancers.

In PCOS, follicular development arrests at the stage of selection of the dominant follicle, at about 7-9 mm in diameter, which may be due in part to abnormal regulation of enzyme functions in the ovary. While the exact mechanism that blocks follicle development is not known, insulin imbalance, abnormalities in the enzymes involved in steroid hormone biosynthesis and genetic predisposition all appear to play a role. Local steroid production in the ovarian follicles is controlled by enzymes expressed in the ovaries that regulate conversion between the steroids (6, 7) (FIG. 1). In PCOS, concentrations of androgens in the follicular fluid (FF) have been shown to be higher than in non-PCOS women (5).

A number of studies have examined the relationship between concentrations of specific steroids in FF from women who have undergone ovarian stimulation protocols in preparation for IVF and association of steroid concentrations with IVF outcome. An increased cortisol/cortisone ratio (8, 9) and lower concentrations of cortisone in FF (8) has been associated with a positive outcome (i.e., successful pregnancy) of IVF in some studies, while others have failed to find any association between cortisone concentrations with IVF outcome (10). Higher concentrations of progesterone and progesterone/estradiol (E2) ratio in FF samples have been associated with positive outcome of IVF in one study (11), while lower progesterone concentrations were associated with positive outcome in another study (12). Higher E2/androstendione and E2/testosterone ratios have also been associated with positive outcome in IVF (13). Due to the variation in reported results from these studies, the association of concentrations of steroids in FF with IVF outcome has remained unclear. Previous studies have not attempted to examine the association between concentrations of multiple steroids and the outcome of IVF.

Furthermore, the information on steroids present in FF and their concentrations in RM women is conflicting. In part this is related to the very limited sample volume of FF that may be obtained from follicles of RM women and the absence of sensitive and specific methods allowing simultaneous quantitative analysis of multiple steroids in such small samples. In previous studies (7-16), measurements of steroids in FF were performed using immunoassays (IA), which may have high cross-reactivity with structurally-related compounds (17), or using gas chromatography mass spectrometry (GC-MS) methods, which are more specific but require larger sample aliquots (18-19). Recent advancements in biological mass spectrometry helped overcome some of the problems associated with poor sensitivity and specificity of immunoassays and has enabled simultaneous accurate quantification of multiple analytes.

Liquid Chromatography-tandem Mass Spectrometry (LC-MS/MS) methods allow high sensitivity detection and accurate quantification of a large number of steroids using a small sample volume (20-25). Increased knowledge about the underlying mechanisms and processes involved in the regulation of the menstrual cycle and ovulation may help to understand anovulatory conditions, such as in PCOS, and help to tailor and fine-tune in vitro fertilization (IVF) regimens. In addition, knowledge of specific steroid profiles which are associated with PCOS and other endocrine disorders may be useful in providing a definitive diagnosis of a specific condition or guiding treatment. Identification of specific steroid profiles in FF associated with outcomes of successful or unsuccessful pregnancy following IVF treatments can also be used for predicting outcomes and selecting oocytes which have a greater probability of resulting in a successful pregnancy in IVF treatments; alternatively oocytes, which are identified as having a low probability of achieving viable pregnancy can be selected for use in generation of embryonic stem cells for related procedures, such as research or therapy.

### SUMMARY OF THE INVENTION

In accordance with the present invention, specific steroid profiles in FF are identified for diagnostic and prognostic use in identifying and treating conditions relating to ovarian function in women. The present invention determines the concentrations of endogenous steroids in FF and describes an association between the patterns of distribution of steroids in FF during the early follicular phase of the menstrual cycle and after ovarian stimulation for in vitro fertilization (IVF), thereby providing means for identifying potential strategies leading to successful outcomes of in vitro fertilization (IVF). The present invention also describes the steroid profiles in ovarian FF samples from women diagnosed with PCOS and in the early follicular phase of regularly menstruating women. The differences in concentrations of steroid hormones, the patterns of their distribution and differences in product/precursor ratios of steroids (illustrating relative enzyme activities), and the associations between concentrations of steroids in the FF and baseline characteristics are determined.

The invention also relates to the use of a steroid profile as a diagnostic method for the identification of deficiencies or defects in one or more steroid synthesis pathway. For example, a low concentration of progesterone relative to the concentration of pregnenolone in FF samples may be indicative of a deficiency of 3βHSD. Thus, the steroid profiles of the invention provide diagnostic methods for identifying abnormal regulation in the steroid biosynthesis pathway. In addition, the identification of defects in the steroid biosynthesis pathway may also be used for selecting an appropriate IVF protocol, to predict outcome of IVF treatment, to select oocytes which are more likely to lead to a viable pregnancy and/or to modify an IVF protocol for improving chances of successful outcome.

Diagnostic testing is more clinically useful when the results are related to an appropriate reference value. Comparing the pattern of distribution of steroids in the FF from PCOS and non-PCOS women provides a method for associating specific steroids or enzyme-regulating conversions that are important for normal ovarian regulation with abnormally regulated enzymes that characterize the follicular arrest in PCOS women.

More particularly, accumulation in the ovaries of a large number of atretic follicles and an excess of androgens are characteristic, but not specific, markers of PCOS. Because of this, PCOS is considered a diagnosis of exclusion, meaning that the diagnosis is generated by the exclusion of other possible diseases causing similar symptoms. It is common practice to base diagnosis of PCOS on patient history, physical examination and semi-specific laboratory tests (e.g., LH-FSH ratio, free and total androgens). The testing is usually performed for the purpose of excluding other diseases which cause symptoms similar to PCOS. In contrast, the present invention identifies steroid profiles in the FF of women with PCOS and provides a comparison to the steroid concentrations observed in FF of RM women, thereby identifying specific biomarkers of PCOS (FIGS. 3-4). Thus, the invention provides a more specific method for direct diagnosis of PCOS based on measurement of biomarkers in ovarian follicular fluid.

The invention also provides steroid response profiles for ovarian stimulation during IVF treatment which allow a physician to choose the most suitable protocol, to select oocytes which are more likely to result in viable pregnancy, or to modify the protocol to obtain, diagnose, or prognose the successful outcome and avoid complications of the therapy or of the procedure as a whole.

The invention provides values of steroid concentrations and ratios of concentrations of steroids in FF from women diagnosed with PCOS and from regularly menstruating women, thereby providing a diagnostic method for certain conditions and determination of appropriate treatment regimens. LC-MS/MS methods are highly sensitive and specific and allow simultaneous measurement of multiple steroids, and are, therefore, suitable methods for better understanding the underlying mechanism and/or processes involved in the regulation of the menstrual cycle, ovulation and anovulation. In addition, the invention provides a diagnostic and/or prognostic method that allows for identification of patients who are more likely to have a successful or unsuccessful outcome in IVF treatment, for selection of oocytes which are more likely to lead to viable pregnancy following IVF treatment, and the tailoring and fine-tuning of IVF-regimens to reach the goal of successful ovulation and pregnancy.

The invention also provides a kit for determining a steroid profile comprising written instructions, at least one composition capable of use as an internal standard, and at least one reference standard. The kit may include a reference standard, wherein a steroid profile from a sample that differs from the reference is indicative of a disease condition or physiological state.

### BRIEF DESCRIPTION OF THE DRAWINGS

FIG. 1 depicts the pathway for biosynthesis of steroids, and the enzymes involved in the pathway;

FIG. 2A illustrates the distribution of median concentrations of steroids in FF of regularly menstruating women from androgen-dominant follicles, where androgen-dominant follicles are defined as having an E2/Te ratio <4 (4);

FIG. 2B illustrates the distribution of median concentrations of steroids in FF of regularly menstruating women from estrogen-dominant follicles, where estrogen-dominant follicles are defined as having an E2/Te ratio >4;

FIG. 3A illustrates the distribution of median concentrations of steroids in FF of healthy women;

FIG. 3B illustrates the distribution of median concentrations of steroids in FF of women diagnosed with PCOS;

FIG. 4 shows ROC curves for six biomarkers of PCOS in FF samples;

FIG. 5 illustrates comparative distributions of concentrations of 17-OH Progesterone (A), 17-OH Pregnenolone (B), Pregnenolone (C) and Total Pregnenolones (D) in subjects with a viable pregnancy and subjects with no viable pregnancy;

FIG. 6 illustrates comparative distributions of concentrations of Estrone (A), Estradiol (B), Estriol (C) and Total Estrogens (D) in subjects with a viable pregnancy and subjects with no viable pregnancy;

FIG. 7 illustrates comparative distributions of concentrations of DHEA (A), Androstenedione (B), hydroxyprogesterone (C) and Total Androgens (D) in subjects with a viable pregnancy and subjects with no viable pregnancy;

FIG. 8 illustrates comparative distributions of concentrations of Cortisone (A), Cortisol (B), 11-Deoxycortisol (C) and Total Glucocoricoids (D) in subjects with a viable pregnancy and subjects with no viable pregnancy;

FIG. 9 illustrates two distinct steroid profiles present within the group with no pregnancy or lost pregnancy outcomes.

### DETAILED DESCRIPTION OF THE INVENTION

A key to the abbreviations used herein is as follows:
A4 Androstenedione
ADF Androgen-dominant follicles
Allopregn Allopregnalone
ANDR Androgen
AUC Area under curve
CV Coefficient of variation
DHEA Dehydroepiandrostenedione
11DC 11 Deoxycortisol
E Cortisone
E1 Estrone
E2 Estradiol
E3 Estriol
EDF Estrogen-dominant follicles
ESI Electrospray ionization
ESTR Estrogens
F Cortisol
FF Follicular fluid
GC-MS Gas chromatography mass spectrometry
17OHP 17-hydroxyprogesterone
17OHPregn 17-hydroxypregnenolone
HPLC High performance liquid chromatography
HSD Hydroxysteroid dehydrogenase
IA Immunoassay
IS Internal standard
IVF In-vitro fertilization
LC-MS/MS Liquid chromatography tandem mass spectrometry
MRM Multiple reaction monitoring
m/z Mass to charge ratio
Pregn Pregnenolone
Prog Progesterone
PCOS Polycystic ovary syndrome
RIA Radioimmunoassay
RM Regularly menstruating
ROC Receiver operating characteristic
SD Standard deviation
SHBG Sex hormone binding globulin
Te Testosterone

As used herein and in the appended claims, the singular forms “a”, “an”, and “the” include plural reference unless the context clearly dictates otherwise. For example, reference to “a steroid” includes a plurality of such steroids, and reference to the “a steroid profile” is a reference to one or more profiles, and so forth.

As used herein, “comprising,” “including,” “having,” “containing,” “characterized by,” and grammatical equivalents thereof, are inclusive or open-ended terms that do not exclude additional, unrecited elements or method steps, but also include the more restrictive terms “consisting of” and “consisting essentially of.”

As used herein, “successful pregnancy” or “viable pregnancy” means the successful implantation of a fertilized ovum such that fetal development and birth are likely to result.

As used herein, “outcome,” when used in association with “in vitro fertilization,” is inclusive of both viability of an oocyte and non-viability of an oocyte for in vitro fertilization. As used herein, “successful outcome of in vitro fertilization” means successful fertilization of an ovum that is suitable for implantation and intrauterine development.

During the last decade, tandem mass spectrometry has become the method of choice for analyzing endogenous steroids. The methods used herein allow accurate quantitation of thirteen steroids from 40 μL of FF. Analysis of these steroids using IA-based methods would require at least a few milliliters of FF, which is a sample size that is unrealistic for follicles during early follicular stage of the menstrual cycle or for follicles of women with PCOS. In addition there are some pitfalls associated with use of immunoassays for analyzing FF samples. Compared to serum, FF has significantly higher concentrations of some of the steroids, and the difference in concentrations may cause cross-reactivity that is not observed in the serum samples (for which IA are typically validated). Another pitfall is related to the need of reducing the concentration of steroids into the range measurable by the IA by diluting the FF. The characteristics of the diluents could alter the binding of proteins thus affecting the observed concentrations in methods not including extraction steps prior to IA. The above problems are not relevant to the mass spectrometry-based methods.

### Example I

### Methods for the Analysis of Steroid Patterns in FF Samples from RM Women

### Participants

Twenty-one regularly menstruating (RM) women of Caucasian decent were recruited for the study. The women attended the hospital for laparoscopic treatment of infertility presumably caused by pelvic adhesions. All women had regular cycles and normal ovaries on pelvic ultrasound examination, were in good general health and had not taken hormonal medication or oral contraceptives during the last three months before inclusion in the study. The study was approved by the Ethics Committees in Donetsk State Medical University (Ukraine) and in Uppsala University (Sweden).

### Collection and Handling of Follicular Fluid Samples

In RM women, FF samples were obtained between days 4 and 7 of the follicular phase of a cycle during laparoscopic adhesiolysis. FF aspirated from ovarian follicles (5-8 mm diameter) was pooled within each subject and centrifuged. Size of the follicles was measured by transvaginal ultrasonography performed during laparoscopic adhesiolysis. The samples were transferred in microcentrifuge tubes and stored at −70° C. until analysis. Clinical and anthropometrics characteristics of participating women are listed in Table 1, below.

TABLE 1
Anthropometric and reproductive characteristics of healthy
women of fertile age (n = 21).

| Variable | Mean ± SD median [range] |
|---|---|
| Age (years) | 28 ± 3.2# |
| Height (cm) | 16.5 ± 6.2 |
| Weight (kg) | 64.8 ± 10.4 |
| BMI (kg/m2) | 23.9 ± 3.8 |
| Parity | 2.1 ± 1.7 [1-8] |
| Average number of menstrual | 12/12 |
| cycles during last 12 months | |
| Menstrual cycle day at follicular | 6 [4-7] |
| fluid sampling | |
| Menstrual cycle length (days) | 28 [21-32] |
| Hirsutism index ## | 3 [1-8] |
| Current smokers | 9/21 |

#range: 21-34 years;
## Modified Ferriman and Gallwey scale

### Reagents and Standards

Testosterone (Te), estrone (E1), 17βE2, 17αE2, estriol (E3), pregnenolone (Pregn), 17 hydroxypregnenolone (17-OHPregn), 17 hydroxyprogesterone (17OHP), 11 deoxycortisol (11DC), cortisol (F), cortisone (E), progesterone (Prog), allopregnalone (Allopregn), hydroxylamine, formic acid, trifluoroacetic acid, dansyl chloride and sodium carbonate were purchased from Sigma Chemical Company (St. Louis, Mo.). Androstenedione (A4), dehydroepi-androsterone (DHEA), dihydrotestosterone (DHT) and androstanedione (A) were purchased from Steraloids Inc. (Newport, R.I.). The internal standards (IS) were deuterium) labeled analogs of the steroids d₃-Te, d₃-Pregn, d₂-11DC, d₈-17OHP, d₃-17OHPregn, d₃-E (Cambridge Isotope Laboratories, Andover, Mass.); and d₄-E1, d₃-E2, d₃-E3 and d₄ Allopregn (CDN Isotopes, Toronto, ON). Methanol, acetonitrile, and methyl-tert-butyl ether (MTBE) were all HPLC grade from VWR (West Chester, Pa.). All other chemicals were of the highest purity commercially available.

### LC-MS/MS Methods

Concentrations of all steroids in FF were determined using. LC-MS/MS based methods (20-25). Estrogens were analyzed as dansyl derivatives (23, 24); ketosteroids were analyzed as oxime derivatives (21-22), cortisol and cortisone were analyzed as non-derivatized (20). The HPLC system consisted of series 1200 HPLC pumps (Agilent, Santa Clare, Calif.); a 10-port switching valve, a vacuum degasser and an autosampler HTC PAL (LEAP Technologies, NC) equipped with a fast wash station. An API 4000 (Applied Biosystems/MDS SCIEX) tandem mass spectrometer was used in the positive ion mode with a TurboIonspray™ ion source. Sample preparation, chromatographic separation conditions, and mass transitions used in the methods have been previously described (20-25) and are summarized in Table 2, below,

TABLE 2
Outline of sample preparation and instrumental analysis for determination of concentrations of steroids in FF samples.

| | | Sample | | | Mass transitions, m/z (Collision energy, V) | |
|---|---|---|---|---|---|---|
| Analyte | IS | preparation | LC column | LC conditions | Quantitative | Qualitative |
| Pregnenolone | d₄-Pr | 20 μL of follicular | Synergy Fusion RP, | Mobile phase: 70% | 332 to 86 (40 V) | 332 to 300 (30 V) |
| (Pr) | | fluid (FF) extracted | 50 × 2 mm, 5 μm | methanol, 30% | | |
| 17-OH-pregnen- | d₃-17OHPr | by SPE, derivatized | (Phenomenex). | formic acid, 5 | 348 to 330 (5 V) | 348 to 312 (20 V) |
| olone (17OHPr) | | with hydroxylamine, | | mM, flow rate | | |
| 17-OH-proges- | d₈-17OHP | derivative extracted | | 250 μL/min | 361 to 124 (45 V) | 361 to 112 (45 V) |
| terone (17OHP) | | with MTBE | | | | |
| 11-deoxy- | d₂-11DC | | | | 377 to 124 (42 V) | 377 to 112 (42 V) |
| cortisol (11DC) | | | | | | |
| Testosterone | d₃-Te | | | | 304 to 124 (40 V) | 304 to 112 (40 V) |
| (Te) | | | | | | |
| DHT | d₃-Te | | | | 304 to 253 (32 V) | 304 to 213 (32 V) |
| DHEA | d₃-Te | | | | 306 to 255 (40 V) | 306 to 215 (40 V) |
| Androstanedioe | d₃-Te | | | | 304 to 286 (30 V) | 304 to 271 (30 V) |
| Androstenedioe | d₃-Te | | | | 317 to 124 (40 V) | 317 to 124 (45 V) |
| Progesterone | d₈-17OHP | | | | 345 to 124 (40 V) | 345 to 112 (40 V) |
| Allopregnan- | d₄-Allo | | | | 334 to 86 (48 V) | 334 to 316 (25 V) |
| olone (Allo) | | | | | | |
| Hydroxy- | d₈-170HP | | | | 304 to 124 (40 V) | 304 to 112 (40 V) |
| progesterone | | | | | and 346 to 124 (40 V) | and 346 to 112 (40 V) |
| Cortisol (F) | d₄-F, | 10 μL of FF | Luna Phenyl-hexyl | Mobile phase: 50% | 363 to 121 (35 V) | 363 to 97 (45 V) |
| Cortisone (E) | d₃-E | extracted with | 50 × 2 mm, 5 μm | methanol; 50% | 361 to 163 (35 V) | 361 to 163 (25 V) |
| | | MTBE, evaporated, | particles | water, 5 mM; flow | | |
| | | reconstituted | (Phenomenex). | rate 300 μL/min | | |
| Estrone (E1) | d₄-E1 | 10 μL of FF, | 2D LC: 1^{st} dimension | Gradient 90% water | 504 to 156 (75 V) | 504 to 171 (45 V) |
| 17α-estradiol | d₃-17βE2 | extracted with | separation C1, | to 50% water (in | 506 to 156 (75 V) | 506 to 171 (45 V) |
| | | MTBE derivatized | 2^{nd} dimension | methanol) | | |
| 17β-estradiol (E2) | d₃-17βE2 | with dansyl chloride | Germini C6 100 × | Gradient 50% water | 506 to 156 (75 V) | 506 to 171 (45 V) |
| Estriol (E3) | d₃-E3 | | 2 mm, 3 μm (both | to 85% water (in | 522 to 156 (75 V) | 544 to 171 (45 V) |
| | | | Phenomenex). | acetonitrile), flow | | |
| | | | | rate 600 μL/min | | |

The quadrupoles Q1 and Q3 were tuned to unit resolution and the mass spectrometer conditions were optimized for maximum signal intensity of each steroid. Two mass transitions were monitored for each steroid and the steroid's IS. Concentrations of each steroid were determined using the primary mass transitions; specificity of the analysis for each steroid in every sample was evaluated by comparing concentrations determined using the primary and secondary mass transitions of each steroid and the steroid's IS (26). Quantitative data analysis was performed using Analyst™ 1.4.2 software (Applied Biosystems/MDS SCIEX). The assays showed within-run variation of less than 10% and between-run variation of less than 12%. Calibration curves were generated with every set of samples using six calibration standards; three quality control samples were included with every set of samples.

Concentrations of steroids in FF fluid of women after ovarian stimulation, obtained using LC-MS/MS methods, were compared to values observed in three studies (13-16) using IA methods and one study using liquid chromatography followed by spectrophotometric detection (14). The comparison of steroid concentrations is shown in Table 3, below. Values obtained by LC-MS/MS methods were usually lower, and in some cases were considerably lower than those obtained by the other techniques, especially for testosterone (e.g., up to 18-fold difference). These differences are likely due to cross-reactivity of IA methods intended for performing measurements in specific matrices (i.e., serum) rather than in FF, and suggest the necessity of using highly specific methods for performing measurements of steroids in FF samples.

TABLE 3
Median values of concentration of steroids in FF samples
collected at oocyte retrieval from women undergoing ovarian stimulation
determined with LC-MS/MS and IA methods, comparing values from
the present study and values reported in published studies.

| | | De Sutter | | Bergh | |
|---|---|---|---|---|---|
| | Present | et al | Andersen | et al | Smitz et al |
| | study | 1991 (14) | 1993 (13) | 1995 145) | 2007 (16) |
| Foll.diam, mm | >15 | Na | >12 | >12 | na |
| Method | LC- | LC- | IA | IA | IA |
| | MS/MS | Spectr. | | | |
| 17OHP | 520 | 460 | | | |
| DHEA | 2.7 | | | 4.8 | |
| A4 | 6.8 | 19.3 | 14.1 | 18.6 | 14.6 |
| Te | 0.3 | | 2.9 | 5.5 | 4.4 |
| E1 | 24 | | | 29 | |
| E2 | 240 | 390 | 594 | 373 | 431 |
| Cortisol | 53 | | | | 188 |
| Cortisone | 12 | | | | 18 |

Conc. are in ng/mL.;
na = data not available;
LC-Spectr = liquid chromatography-spectrophotometry

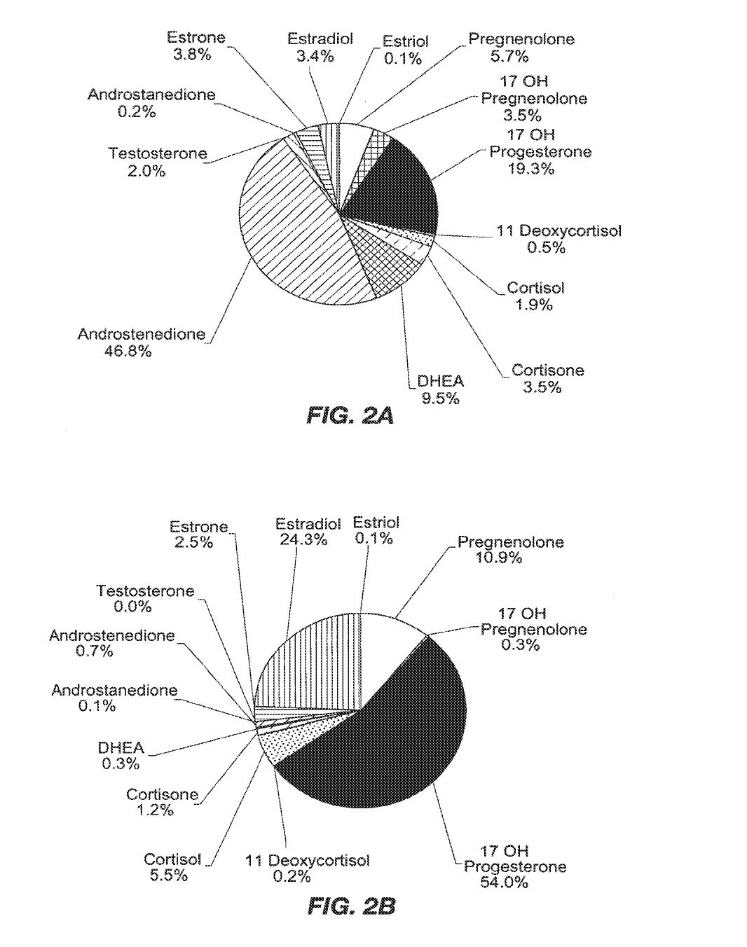

The distribution pattern of steroid concentrations in androgen-dominant follicles (n=13) and estrogen dominant follicles (n=8) was also analyzed, as illustrated in FIG. 2A and FIG. 213. Androgen-dominant follicles (ADF) were defined as having an E2/Te ratio <4, and estrogen-dominant follicles (EDF) were defined as follicles with the E2/Te ratio >4 (26). Steroids for which significant differences were demonstrated between ADF and EDF are given in Table 4, below. Compared to ADF, EDF had significantly higher concentration of E2, significantly higher E2/E1-ratio and significantly lower concentrations of A4 and Te, (Table 4). In ADF, A4 was the dominating steroid (56.4%), followed by 17-OHP and DHEA. In EDF, A4 was also the dominating steroid (30.8%), followed by 17-OHP and E2 (FIG. 2).

TABLE 4
Variables showing significant differences between FF samples
from androgen dominant (ADF) and estrogen dominant (EDF) follicles
from RM women.

| | ADF | | EDF | |
|---|---|---|---|---|
| N | 13 | | 8 | |
| A4 | 590 | (330-890) | 300 | (180-410)** |
| Te | 25 | (15-54) | 7.5 | (6.0-21)** |
| E2 | 14 | (2.0-43) | 190 | (33-490)*** |
| E1 | 22 | (3.3-97.1) | 83 | (15.5-139.9)* |
| E2/E1-ratio | 0.42 | (0.15-2.44) | 2.16 | (0.81-6.64)** |

Conc. in ng/mL; Median (5th-95th percentile)
Significance of differences between the groups denoted by:
*p < 0.05,
**p < 0.01, and
***p < 0.001, respectively.

The concentrations of various steroids from FF samples taken from RM women were determined and are shown in Table 5, below.

TABLE 5
Concentrations of steroids in FF samples of RM women
measured by LC-MS/MS. Median (5th-95th percentile).

| | RM women | |
|---|---|---|
| Number | 21 | |
| Pregnenolone (Pregn) | 52 | (16-89) |
| 17OH pregnenolone (17OH Pregn) | 32 | (4.4-60) |
| 17OH progesterone (17OHP) | 180 | (65-310) |
| 11 deoxycortisol (11DC) | 4.1 | (1.8-6.6) |
| Cortisol (F) | 17 | (3.9-38) |
| Cortisone (E) | 32 | (19-47) |
| Dehydroepiandrosterone (DHEA) | 86 | (34-190) |
| Adrostenedione (A4) | 420 | (200-830) |
| Testosterone (Te) | 18 | (6.2-43) |
| Androstanedione (A) | 2.0 | (0.6-6.2) |
| Androgens total | 534,013 | (252-997) |
| Estrone (E1) | 34 | (3.3-140) |
| Estradiol (E2) | 31 | (2.6-302)^{#} |
| Estriol (E3) | 0.47 | (0.1-2.3)^{#} |
| Estrogens total | 66 | (11-388)^{#} |
| F/E ratio | 0.55 | (0.14-1.19) |
| E2/E1 ratio | 0.66 | (0.15-3.51)^{#} |
| E2/Te ratio | 1.5 | (0.12-42)^{#} |

^{#}one result was excluded as outlier (using Mahalanobis test).
Concentrations in ng/mL.

### Example II

### Steroid Profiles in FF from Women with and without PCOS

### Participants

Study subjects were recruited and investigated at the Donetsk Regional Center of Mother and Child Care, Donetsk, Ukraine. FF from 27 women with PCOS and 21 regularly cycling women without PCOS were included in this study. The diagnosis of PCOS was based on amenorrhea or oligomenorrhea (<10 cycles per year), a characteristic ovarian image on ultrasound examination (≧10 small follicles per plane, in association with a marked ovarian stroma) (27). Hirsutism, was assessed by a modified version of the protocol used by Ferriman and Gallwey (28) and women with a score of ≧8 were considered clinically hirsute. BMI was calculated as weight (kg) divided by height (m) squared. All the ultrasound examinations were performed transabdominally or transvaginally (3.5 and 5 MHz sector probe, respectively; Kranzbühler GmBH, Germany). The PCOS patients were treated for infertility by ovarian wedge resection and FF was collected during that surgery.

Control subjects were women with infertility presumably caused by pelvic adhesions. These women had regular menstrual cycles and normal ovaries on pelvic ultrasound examination. All subjects were in good general health and had not taken hormonal medication or oral contraceptives during the preceding three months prior to inclusion in the study. Ultrasound images from women diagnosed with PCOS and controls were blindly evaluated by two independent Swedish gynecological ultrasound experts.

Sampling was performed between days 3 and 7 in the follicular phase in RM women (controls) and at any day in oligo-/amenorrheic patients. FF from women diagnosed with PCOS and FF from follicles having a diameter of 5-8 mm in control women were pooled within each subject and centrifuged. Follicle size was measured by transvaginal ultrasonography performed during laparoscopic surgery (wedge resection for PCOS women) or adhesiolysis (controls). The samples were kept frozen at below −20° C. until used for analysis.

The reagents and standards for FF analysis were the same as described in Example 1, above. Likewise, the LC-MS/MS methods were the same as described above in Example 1.

Baseline comparisons between the study groups (PCOS and RM women) were assessed using non-parametric Wilcoxon two-group tests for continuous variables and Chi-square test. Associations between variables were accessed using the Spearman rank correlation test. Multiple logistic regression analysis was used to explore the putative independent effects of measured hormones and product/precursor ratios (enzyme activities) with regard to presence of PCOS. Receiver Operating Characteristic (ROC) curves, were plotted for evaluation of steroids biomarkers of PCOS in FF samples. For every statistically significant result cited, the p value was less than 0.05, unless otherwise specified. Statistical analyses were performed using the JMP software (SAS Institute Inc., NC, USA). Values of steroid concentrations and the ratios of steroid concentration are expressed as median and range, unless otherwise stated.

Clinical data and hormone concentrations for individual study participants are given in Table 6, below,

TABLE 6

| | | | | | | Sum ocf | | | | | | | | |
|---|---|---|---|---|---|---|---|---|---|---|---|---|---|---|
| | | | | | | concentra- | | Total | | | | | | |
| Sample | | 17 OH | | Total | Total | tions (A4 + | | Pregnen- | Total | | | Total | | |
| ID | Group | Pregn | E1 | ANDR | steroids | DHEA + 17OHP) | A4 | olones | GC | DHEA | E3 | ESTR | 11DC | Cortiso |
| 37 | Normal | 4.38 | 34.1 | 1104.2 | 1142.7 | 1160.2 | 992 | 20.78 | 41.56 | 34.2 | 0.82 | 49.1 | 5.06 | 3.9 |
| 38 | Normal | 48.4 | 55.1 | 997.3 | 1100.8 | 1165 | 829 | 106.5 | 61.17 | 132 | 0.494 | 77.4 | 5.17 | 28.1 |
| 39 | Normal | 82.1 | 143 | 454.8 | 679.9 | 629 | 251 | 170.7 | 43.45 | 197 | 1.01 | 216.8 | 3.25 | 17.5 |
| 45 | Normal | 4.41 | 3.24 | 627.6 | 635.3 | 647.4 | 562 | 13.61 | 35.01 | 26.8 | 1.16 | 5.6 | 3.37 | 3.84 |
| 46 | Normal | 13.3 | 134 | 483.2 | 630.5 | 696.7 | 398 | 53.9 | 60.35 | 61.7 | 3.67 | 439.7 | 5.05 | 13.3 |
| 53 | Normal | 36.7 | 31.1 | 650.9 | 718.7 | 858.4 | 549 | 85.7 | 60.15 | 81.4 | 0.721 | 85.8 | 4.85 | 21.6 |
| 54 | Normal | 15.3 | 3.26 | 354.9 | 373.5 | 402 | 289 | 67.2 | 87.3 | 48.5 | 0.451 | 15.1 | 1.6 | 57 |
| 71 | Normal | 54.5 | 66.6 | 758.3 | 879.4 | 975 | 599 | 121.8 | 80.91 | 128 | 0.402 | 102.5 | 5.51 | 32.7 |
| 73 | Normal | 49.6 | 11.8 | 511.2 | 572.6 | 677 | 384 | 114.6 | 56.08 | 118 | 0.378 | 51.1 | 3.08 | 21.5 |
| 89 | Normal | 59.9 | 18.1 | 734.3 | 812.3 | 884 | 531 | 129.1 | 56.19 | 185 | 0.0809 | 20.7 | 3.59 | 20.5 |
| 91 | Normal | 43.5 | 21 | 486.0 | 550.5 | 618 | 353 | 109.8 | 89.58 | 117 | 0.34 | 54.8 | 2.58 | 38.3 |
| 92 | Normal | 11.5 | 22.5 | 267.7 | 301.7 | 371 | 220 | 43.5 | 36.24 | 39 | 0.148 | 53.1 | 1.84 | 16.9 |
| 107 | Normal | 18.3 | 11.2 | 806.2 | 835.7 | 878.9 | 700 | 41.3 | 56.6 | 62.9 | 0.367 | 18.7 | 4.2 | 16.8 |
| 108 | Normal | 14.8 | 70.6 | 252.1 | 337.5 | 557.3 | 198 | 114.3 | 36.07 | 46.3 | 10.9 | 669.5 | 6.57 | 10.5 |
| 117 | Normal | 31.9 | 80.3 | 534.0 | 646.2 | 732.8 | 424 | 87.7 | 53.67 | 85.8 | 2.11 | 197.4 | 3.87 | 10 |
| 119 | Normal | 28.8 | 6.25 | 526.3 | 561.4 | 595.5 | 420 | 54.3 | 61.96 | 88.7 | 0.091 | 10.5 | 2.96 | 32 |
| 120 | Normal | 35.8 | 65.3 | 862.5 | 963.6 | 1013 | 716 | 95.3 | 46.61 | 101 | 0.243 | 92.6 | 6.01 | 12 |
| 121 | Normal | 38.5 | 143 | 774.7 | 956.2 | 1061 | 639 | 119.2 | 53.86 | 117 | 0.694 | 171.4 | 6.56 | 11.5 |
| 122 | Normal | 45.5 | 22.3 | 786.6 | 854.4 | 895 | 594 | 96.9 | 64.9 | 163 | 0.0997 | 25.7 | 4.1 | 13.8 |
| 123 | Normal | 17.9 | 84.8 | 220.8 | 323.5 | 387.3 | 164 | 55.7 | 36.76 | 48.3 | 2.26 | 385.1 | 2.51 | 5.95 |
| 124 | Normal | 26.8 | 124 | 433.8 | 584.6 | 638.9 | 343 | 72.5 | 59.09 | 83.9 | 1.54 | 381.5 | 4.79 | 13 |
| 6 | PCOS | 14.3 | 2.1 | 239.7 | 415.8 | 274.3 | 193 | 27.4 | 91.29 | 36.2 | 1.67 | 12.3 | 1.79 | 59.8 |
| 8 | PCOS | 47.7 | 9.51 | 990.9 | 1386.8 | 1130 | 807 | 95.8 | 104.31 | 147 | 0.482 | 19.8 | 6.21 | 57.8 |
| 9 | PCOS | 64.6 | 5.54 | 1170.5 | 1558.7 | 1303 | 898 | 119.4 | 61.45 | 234 | 0.492 | 36.4 | 5.35 | 16.1 |
| 20 | PCOS | 71.8 | 26.2 | 1177.3 | 1620.8 | 1315 | 913 | 139.7 | 90.27 | 220 | 0.374 | 31.5 | 5.97 | 40.2 |
| 29 | PCOS | 79.9 | 7.66 | 1145.2 | 1728.3 | 1393 | 928 | 147.2 | 124.3 | 177 | 0.32 | 23.6 | 8.1 | 66.7 |
| 32 | PCOS | 35.5 | 5.41 | 1300.6 | 1589.9 | 1404 | 1100 | 64.9 | 59.81 | 153 | 0.274 | 13.5 | 6.01 | 15.9 |
| 35 | PCOS | 43.1 | 3.13 | 801.8 | 1058.7 | 890 | 631 | 66 | 70.32 | 142 | 0.168 | 3.6 | 5.32 | 17.8 |
| 51 | PCOS | 48.1 | 10.8 | 606.4 | 967.9 | 747.5 | 499 | 107.4 | 69.64 | 86.5 | 0.696 | 22.5 | 4.14 | 32.2 |
| 52 | PCOS | 71.8 | 4.31 | 862.9 | 1339.3 | 1090 | 722 | 161.9 | 49.17 | 120 | 0.428 | 17.3 | 3.87 | 20.5 |
| 56 | PCOS | 82.4 | 11.1 | 1526.5 | 2043.6 | 1731 | 1240 | 160.2 | 69.07 | 234 | 0.54 | 30.8 | 7.37 | 29 |
| 57 | PCOS | 53.1 | 24.7 | 863.8 | 1325.6 | 1048 | 657 | 112.8 | 61.23 | 176 | 0.119 | 72.8 | 4.63 | 13.3 |
| 63 | PCOS | 63 | 23.6 | 851.4 | 1243.9 | 990 | 646 | 118.6 | 70.85 | 171 | 0.0909 | 30.0 | 4.35 | 23.3 |
| 65 | PCOS | 96.5 | 17.1 | 1579.2 | 2122.4 | 1807 | 1270 | 175 | 66.72 | 263 | 0.416 | 27.5 | 9.42 | 12.6 |
| 72 | PCOS | 66.8 | 11.2 | 851.0 | 1424.1 | 1108 | 688 | 144.9 | 88.43 | 140 | 0.465 | 59.8 | 5.43 | 32 |
| 79 | PCOS | 66.8 | 8.24 | 1025.1 | 1434.6 | 1237 | 828 | 109.6 | 54.7 | 176 | 0.133 | 12.3 | 5 | 17.3 |
| 80 | PCOS | 68.5 | 9.2 | 841.2 | 1259.2 | 1041 | 679 | 121.4 | 57.07 | 139 | 0.113 | 16.5 | 5.77 | 14.9 |
| 82 | PCOS | 72.8 | 35.6 | 1285.2 | 1921.2 | 1523 | 1040 | 132.1 | 50.54 | 213 | 2.75 | 183.4 | 7.48 | 9.16 |
| 84 | PCOS | 103 | 12.2 | 1578.0 | 2382.9 | 2016 | 1310 | 193.8 | 104.5 | 225 | 0.184 | 25.6 | 15.5 | 31.5 |
| 85 | PCOS | 124 | 25.4 | 1348.3 | 2505.7 | 2006 | 1100 | 255 | 153.4 | 210 | 0.387 | 53.0 | 10.6 | 89.6 |
| 87 | PCOS | 61.2 | 6.83 | 883.1 | 1243.1 | 1022 | 698 | 114.2 | 65.35 | 154 | 0.145 | 10.5 | 4.05 | 19.1 |
| 90 | PCOS | 90.1 | 7.08 | 998.8 | 1411.6 | 1181 | 769 | 148 | 49.72 | 206 | 0.0886 | 9.1 | 4.92 | 16.1 |
| 96 | PCOS | 48.9 | 36.4 | 992.3 | 1526.2 | 1167 | 810 | 102.2 | 49.89 | 130 | 0.318 | 154.7 | 6.19 | 5.7 |
| 97 | PCOS | 40.7 | 0 | 920.5 | 1272.3 | 1068 | 769 | 84.8 | 94.06 | 126 | 0 | 0.0 | 5.66 | 32.1 |
| 101 | PCOS | 37.5 | 10.4 | 529.5 | 821.6 | 631.1 | 414 | 68.7 | 80.48 | 96.1 | 0.066 | 22.0 | 3.68 | 49.1 |
| 109 | PCOS | 35.6 | 22.5 | 681.0 | 1010.6 | 786 | 515 | 75.2 | 96.88 | 141 | 0.191 | 27.5 | 4.28 | 35.2 |
| 110 | PCOS | 75.5 | 16.7 | 1254.9 | 1817.4 | 1558 | 1090 | 125.5 | 67.4 | 134 | 0.162 | 35.6 | 10.1 | 16.6 |
| 112 | PCOS | 59.1 | 21.1 | 508.1 | 859.8 | 619 | 317 | 123.3 | 82.11 | 181 | 0.119 | 25.2 | 2.11 | 40.1 |

| | | | | | | Total | Ratio | Ratio Total | Ratio | Ratio | | | Ratio |
|---|---|---|---|---|---|---|---|---|---|---|---|---|---|
| Sample | | Andro- | | | 17 | Pro- | 11DC/ | ESTR/Total | DHEA/ | Cortisol/ | Ratio | Ratio | 17OH Pregn/ |
| ID | Cortisone | stanediol | E2 | Pregn | OH P | gestines | 17OHP | ANDR | 17OHP | Cortisone | E2/E1 | E2/Te | Pregn |
| 37 | 32.6 | 7 | 14 | 16.4 | 134 | 134 | 0.038 | 0.04 | 7.81 | 0.12 | 0.42 | 0.201 | 0.27 |
| 38 | 27.9 | 3 | 22 | 58.1 | 204 | 204 | 0.025 | 0.08 | 2.73 | 1.01 | 0.40 | 0.661 | 0.83 |
| 39 | 22.7 | 1 | 73 | 88.6 | 181 | 181 | 0.018 | 0.48 | 2.40 | 0.77 | 0.51 | 11.761 | 0.93 |
| 45 | 27.8 | 5 | 1 | 9.2 | 58.6 | 58.6 | 0.058 | 0.01 | 6.08 | 0.14 | 0.36 | 0.034 | 0.48 |
| 46 | 42 | 5 | 302 | 40.6 | 237 | 237 | 0.021 | 0.91 | 4.64 | 0.32 | 2.25 | 16.413 | 0.33 |
| 53 | 33.7 | 2 | 54 | 49 | 228 | 228 | 0.021 | 0.13 | 2.22 | 0.64 | 1.74 | 2.842 | 0.75 |
| 54 | 28.7 | 2 | 11 | 51.9 | 64.5 | 64.5 | 0.025 | 0.04 | 3.17 | 1.99 | 3.50 | 0.722 | 0.29 |
| 71 | 42.7 | 6 | 36 | 67.3 | 248 | 248 | 0.022 | 0.14 | 2.35 | 0.77 | 0.53 | 1.414 | 0.81 |
| 73 | 31.5 | 1 | 39 | 65 | 175 | 175 | 0.018 | 0.10 | 2.38 | 0.68 | 3.30 | 4.518 | 0.76 |
| 89 | 32.1 | 2 | 3 | 69.2 | 168 | 168 | 0.021 | 0.03 | 3.09 | 0.64 | 0.14 | 0.159 | 0.87 |
| 91 | 48.7 | 2 | 34 | 66.3 | 148 | 148 | 0.017 | 0.11 | 2.69 | 0.79 | 1.60 | 2.376 | 0.66 |
| 92 | 17.5 | 1 | 31 | 32 | 112 | 112 | 0.016 | 0.20 | 3.39 | 0.97 | 1.36 | 3.756 | 0.36 |
| 107 | 35.6 | 3 | 7 | 23 | 116 | 116 | 0.036 | 0.02 | 3.44 | 0.47 | 0.63 | 0.176 | 0.80 |
| 108 | 19 | 1 | 588 | 99.5 | 313 | 313 | 0.021 | 2.66 | 3.13 | 0.55 | 8.33 | 85.465 | 0.15 |
| 117 | 39.8 | 2 | 115 | 55.8 | 223 | 223 | 0.017 | 0.37 | 2.69 | 0.25 | 1.43 | 5.157 | 0.57 |
| 119 | 27 | 2 | 4 | 25.5 | 86.8 | 86.8 | 0.034 | 0.02 | 3.08 | 1.19 | 0.66 | 0.266 | 1.13 |
| 120 | 28.6 | 3 | 27 | 59.5 | 196 | 196 | 0.031 | 0.11 | 2.82 | 0.42 | 0.42 | 0.635 | 0.60 |
| 121 | 35.8 | 1 | 28 | 80.7 | 305 | 305 | 0.022 | 0.22 | 3.04 | 0.32 | 0.19 | 1.539 | 0.48 |
| 122 | 47 | 3 | 3 | 51.4 | 138 | 138 | 0.030 | 0.03 | 3.58 | 0.29 | 0.15 | 0.124 | 0.89 |
| 123 | 28.3 | 3 | 298 | 37.8 | 175 | 175 | 0.014 | 1.74 | 2.70 | 0.21 | 3.51 | 49.750 | 0.47 |
| 124 | 41.3 | 1 | 256 | 45.7 | 212 | 212 | 0.023 | 0.88 | 3.13 | 0.31 | 2.06 | 41.626 | 0.59 |
| 6 | 29.7 | 1 | 9 | 13.1 | 45.1 | 45.1 | 0.040 | 0.05 | 2.53 | 2.01 | 4.08 | 0.946 | 1.09 |
| 8 | 40.3 | 7 | 10 | 48.1 | 176 | 176 | 0.035 | 0.02 | 3.08 | 1.43 | 1.03 | 0.323 | 0.99 |
| 9 | 40 | 5 | 30 | 54.8 | 171 | 171 | 0.031 | 0.03 | 3.62 | 0.40 | 5.49 | 0.913 | 1.18 |
| 20 | 44.1 | 5 | 5 | 67.9 | 182 | 182 | 0.033 | 0.03 | 3.06 | 0.91 | 0.19 | 0.126 | 1.06 |
| 29 | 49.5 | 6 | 16 | 67.3 | 288 | 288 | 0.028 | 0.02 | 2.22 | 1.35 | 2.04 | 0.455 | 1.19 |
| 32 | 37.9 | 9 | 8 | 29.4 | 151 | 151 | 0.040 | 0.01 | 4.31 | 0.42 | 1.45 | 0.202 | 1.21 |
| 35 | 47.2 | 3 | 0 | 22.9 | 117 | 117 | 0.045 | 0.00 | 3.29 | 0.38 | 0.08 | 0.010 | 1.88 |
| 51 | 33.3 | 4 | 11 | 59.3 | 162 | 162 | 0.026 | 0.04 | 1.80 | 0.97 | 1.02 | 0.636 | 0.81 |
| 52 | 24.8 | 5 | 13 | 90.1 | 248 | 248 | 0.016 | 0.02 | 1.67 | 0.83 | 2.92 | 0.792 | 0.80 |
| 56 | 32.7 | 10 | 19 | 77.8 | 257 | 257 | 0.029 | 0.02 | 2.84 | 0.89 | 1.73 | 0.456 | 1.06 |
| 57 | 43.3 | 4 | 48 | 59.7 | 215 | 215 | 0.022 | 0.08 | 3.31 | 0.31 | 1.94 | 1.798 | 0.89 |
| 63 | 43.2 | 3 | 6 | 55.6 | 173 | 173 | 0.025 | 0.04 | 2.71 | 0.54 | 0.27 | 0.200 | 1.13 |
| 65 | 44.7 | 4 | 10 | 78.5 | 274 | 274 | 0.034 | 0.02 | 2.73 | 0.28 | 0.58 | 0.239 | 1.23 |
| 72 | 51 | 2 | 48 | 78.1 | 280 | 280 | 0.019 | 0.07 | 2.10 | 0.63 | 4.29 | 2.346 | 0.86 |
| 79 | 32.4 | 1 | 4 | 42.8 | 233 | 233 | 0.021 | 0.01 | 2.63 | 0.53 | 0.47 | 0.198 | 1.56 |
| 80 | 36.4 | 2 | 7 | 52.9 | 223 | 223 | 0.026 | 0.02 | 2.03 | 0.41 | 0.78 | 0.339 | 1.29 |
| 82 | 33.9 | 2 | 145 | 59.3 | 270 | 270 | 0.028 | 0.14 | 2.93 | 0.27 | 4.07 | 4.866 | 1.23 |
| 84 | 57.5 | 5 | 13 | 90.8 | 481 | 481 | 0.032 | 0.02 | 2.18 | 0.55 | 1.08 | 0.346 | 1.13 |
| 85 | 53.2 | 11 | 27 | 131 | 696 | 696 | 0.015 | 0.04 | 1.69 | 1.68 | 1.07 | 0.978 | 0.95 |
| 87 | 42.2 | 4 | 4 | 53 | 170 | 170 | 0.024 | 0.01 | 2.52 | 0.45 | 0.52 | 0.128 | 1.15 |
| 90 | 28.7 | 2 | 2 | 57.9 | 206 | 206 | 0.024 | 0.01 | 2.29 | 0.56 | 0.27 | 0.088 | 1.56 |
| 96 | 38 | 5 | 118 | 53.3 | 227 | 227 | 0.027 | 0.16 | 2.66 | 0.15 | 3.24 | 2.500 | 0.92 |
| 97 | 56.3 | 0 | 0 | 44.1 | 173 | 173 | 0.033 | 0.00 | 3.10 | 0.57 | | 0.000 | 0.92 |
| 101 | 27.7 | 3 | 12 | 31.2 | 121 | 121 | 0.030 | 0.04 | 2.56 | 1.77 | 1.11 | 0.697 | 1.20 |
| 109 | 57.4 | 2 | 5 | 39.6 | 130 | 130 | 0.033 | 0.04 | 3.96 | 0.61 | 0.21 | 0.213 | 0.90 |
| 110 | 40.7 | 5 | 19 | 50 | 334 | 334 | 0.030 | 0.03 | 1.77 | 0.41 | 1.12 | 0.719 | 1.51 |
| 112 | 39.9 | 1 | 4 | 64.2 | 121 | 121 | 0.017 | 0.05 | 3.06 | 1.01 | 0.19 | 0.468 | 0.92 |

Women with PCOS had higher BMI values, serum testosterone, Te/SHBG-ratio and a hirsutism index compared to RM women, as shown in Table 7, below.

TABLE 7
Anthropometric and reproductive characteristics of PCOS
women and RM women of fertile age.

| | PCOS | Control |
|---|---|---|
| | (n = 27) | (n = 21) |
| | mean ± SD | mean ± SD |
| Variable | median [range] | median [range] |
| Age (years) | 25 ± 3.5^{#,b} | 28 ± 3.2^{##} |
| Height (cm) | 164 ± 6.4 | 165 ± 6.2 |
| Weight (kg) | 73.5 ± 14.9 | 64.8 ± 10.4 |
| BMI (kg/m²) | 27.2 ± 5.2^{b} | 23.9 ± 3.8 |
| Parity (n) | 1.4 ± 0.9 | 2.1 ± 1.7 |
| Average number of menstrual cycles | 6/12 [0-9] | 12/12 |
| during last 12 months | | |
| Menstrual cycle day of follicular fluid | na | 6 [4-7] |
| sampling | | |
| Menstrual cycle length (days) | na | 28 [21-32] |
| Serum SHBG (nmol/L) | 42.8 ± 31 | 67.0 ± 27 |
| Hirsutism index^{###} | 9 [6-24]^{c} | 3 [1-8] |
| Serum Testosterone (nmol/L) | 2.69 ± 1.2^{b} | 1.6 ± 0.7 |
| Serum T/SHBG | 0.11 ± 0.2^{c} | 0.03 ± 0.02 |
| Current smokers (n) | 9/27 | 9/21 |

^{#}range: 21-34 years;
^{##}range: 19-32 years;
^{###}Modified Ferriman and Gallwey scale;
^{a}p < 0.05,
^{b}p < 0.01,
^{c}p < 0.001

Comparison of Median Values in PCOS Vs. RM Women

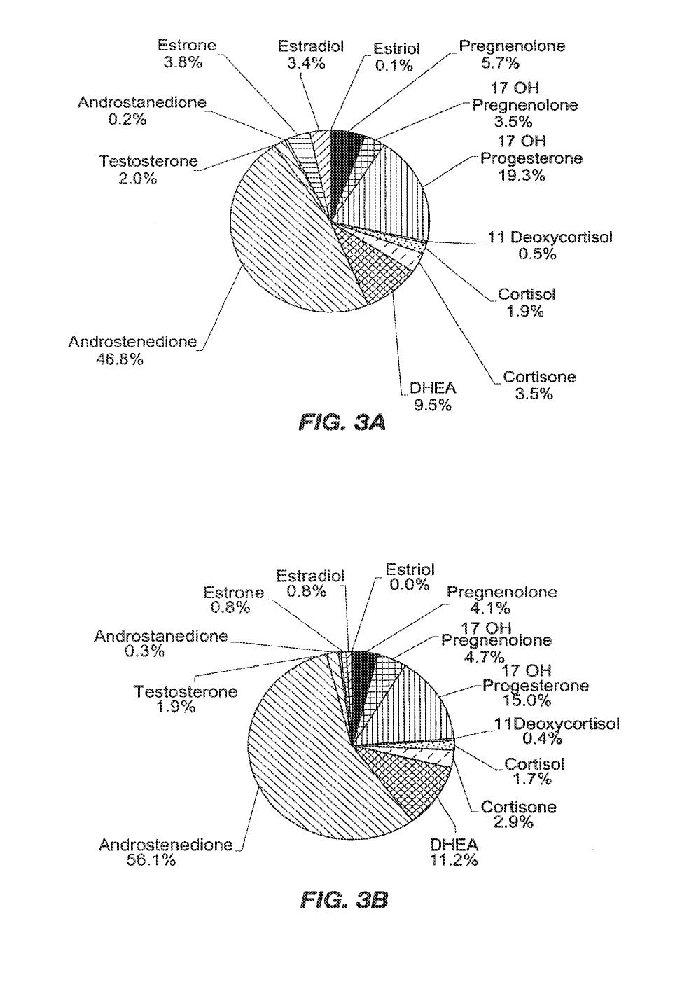

FIG. 3 shows pie diagrams of distribution of median concentrations of measured steroids in FF of RM women (A) and FF of women diagnosed with PCOS(B). In FF from women diagnosed with PCOS, as compared to FF from RM women, concentrations of total androgens were significantly higher (p<0.0001), whereas concentrations of total estrogens (p<0.01) and the ratio of total-ESTR/total-ANDR (p<0.001) were significantly lower. All of these tests remained statistically significant after adjustment for differences in BMI, as set forth in Table 8, below. In addition, in FF of women diagnosed with PCOS, concentrations of 11 deoxycortisol, DHEA, 17 hydroxypregnenolone, androstenedione, testosterone, androstandione, cortisol and cortisone were significantly higher and concentrations of E1, E2 and E3 were significantly lower compared to samples from RM women (Table 8). In PCOS women, BMI was negatively associated with FF concentrations of total estrogens (r=−0.53; p=0.006), 17OHProg; (−0.40; 0.04), and E2 (−0.57; 0.003) and marginally associated with E2/E1 ratio (−0.38; 0.056). Hirsutism index was positively associated with FT concentrations of Te (0.51; 0.006). In regularly menstruating women, BMI was negatively associated with concentration of Pregn (−0.51; 0.018),

TABLE 8
Median concentration of steroids in FF (ng/mL) of PCOS
women and RM women.

| | PCOS | RM | PCOS vs. | |
|---|---|---|---|---|
| | N = 27 | N = 21 | RM | P value* |
| Estrone | 11.0 | 34.1 | − | 0.0016 |
| Estradiol | 10.5 | 30.5 | − | 0.032 |
| Estriol | 0.3 | 0.5 | − | 0.028 |
| Dehydroepiandrosterone | 154.0 | 85.8 | + | <0.0001 |
| 17 hydroxypregnenolone | 64.6 | 31.9 | + | <0.0001 |
| Androstenedione | 769.0 | 424.0 | + | 0.0003 |
| Testosterone | 26.7 | 18.0 | + | 0.024 |
| Androstanedione | 3.6 | 2.0 | + | 0.024 |
| 17 hydroxyprogesterone | 206.0 | 175.0 | + | 0.17 |
| Pregnenolone | 55.6 | 51.9 | + | 0.49 |
| Total androgens | 991.0 | 534.0 | + | <0.0001† |
| Total estrogens | 25.4 | 77.4 | − | <0.006‡ |
| Ratio total estrogens/ | 0.028 | 0.11 | − | 0.0004§ |
| total androgens | | | | |
| 11 deoxycortisol | 5.4 | 4.1 | + | 0.007 |
| Cortisol | 23.3 | 16.8 | + | 0.030 |
| Cortisone | 40.3 | 32.1 | + | 0.004 |

*Non-parametric test (Wilcoxon test);
†adj for BMI, p < 0.0001:
‡Adj for BMI: p < 0.005;
§Adj for BMI: p < 0.01.

### Multiple Logistic Regression Analysis and ROC Analysis

Among the three estrogens tested, E1 was strongly associated with the presence PCOS. When tested alone, E1 yielded AUC=0.77; p=0.009. The association was even stronger than for the total concentration of estrogens. Among the pregnenolones tested, 17OHPregn had the strongest, significant and independent association with PCOS (p=0.0491), followed by Pregn (p=0.061), 17OHPregn (AUC=0.84; p=0.0007) and total ANDR (AUC=0.84; p=0.0010). When evaluated in the same model, E1 and 17OHPregn yielded an AUC of 0.95, and both steroids had significant independent effects, although it was stronger for 17OHPregn; p=0.031 and p=0.0026, respectively. Total ANDR and total ESTR, when included in the same model, yielded an AUC=0.87; both being independent predictors but a stronger relationship was observed for total ANDR, p=0.0044 and p=0.044, respectively.

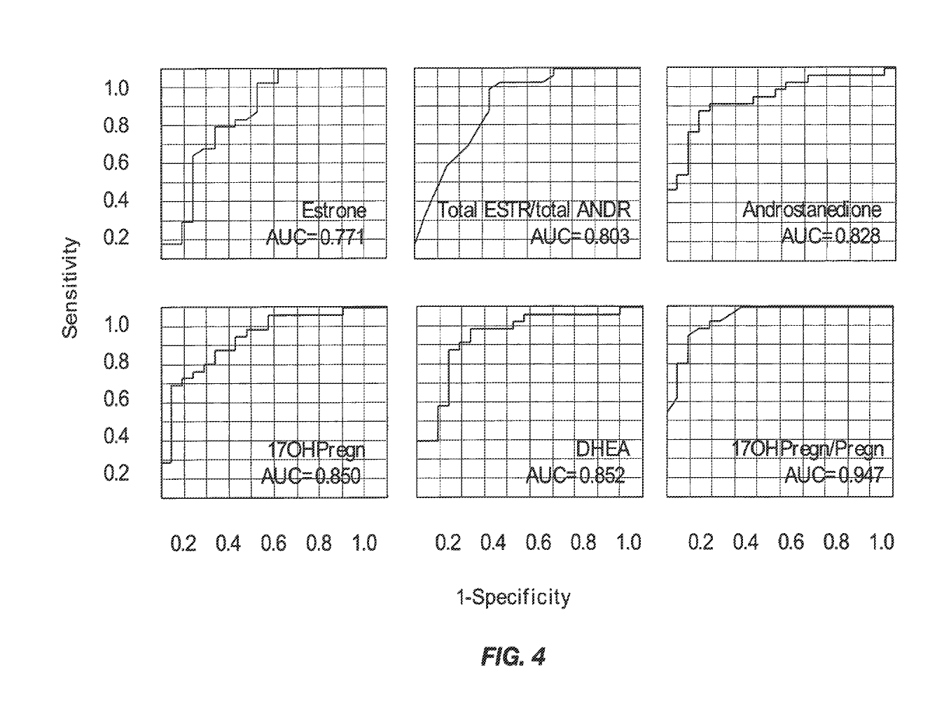

FIG. 4 shows examples of ROC curves for potential steroid biomarkers of PCOS identified herein (only markers with AUC>0.75 are shown). The greatest sensitivity and specificity out of the identified potential biomarkers was the ratio of 17OHPregn/Pregn, followed by concentrations of DHEA, 17OHPregn, androstanedione, the ratio of total estrogens/total androgens and the concentration of estrone. The predictive ability of the biomarkers for determination of PCOS improves when they are used in combination. Thus, the invention includes use of individual biomarkers, ratios of concentrations of the steroid biomarkers, and all combinations of the steroid biomarkers.

### Comparison of the Ratios of Concentrations of Steroid Products/Precursors in the Pathway

Comparison of the product/precursor ratios, as markers of the enzyme activities in the ovarian follicles, as shown in Table 9, below, showed that women with PCOS had a higher activity of CYP17-linked enzymes, favoring higher concentrations of 17OHPregn and A4. In addition, ratios of E1/A4 and E2/Te were five times and 3 times lower, respectively, in PCOS women, indicating a reduced ovarian activity of CYP19-linked enzymes (aromatase).

TABLE 9
Ratio of concentrations of steroids product/precursors of the
pathway values for the groups used as markers of enzyme activities
in PCOS and non-PCOS women.

| | Steroid product/precursor | | Control |
|---|---|---|---|
| | concentration | PCOSN = | women |
| Enzyme | ratios | 27 | N = 21 |
| 3βHSD | 17OHProg/17OHPregn | 3.45^{b} | 6.21 |
| CYP21 | 11DC/17OHProg | 0.028^{0.06} | 0.022 |
| CYP11 | F/11DC | 4.72 | 4.0 |
| 11βHSD type 1 and 2 | E/F | 1.78 | 1.81 |
| CYP17 | DHEA/17OHPregn | 2.66^{a} | 3.08 |
| CYP17 | A4/17OHProg | 3.73^{a} | 2.41 |
| CYP17 | 17OHPregn/Pregn | 1.13^{c} | 0.60 |
| 3βHSD | A4/DHEA | 4.89 | 4.92 |
| 17HSD3 | Te/A4 | 0.035 | 0.040 |
| CYP19 | E1/A4 | 0.014^{c} | 0.067 |
| CYP19 | E2/Te | 0.455^{a} | 1.54 |
| 17βHSD1 type 1 and 2 | E2/E1 | 1.08 | 0.66 |

*Non-parametric test (Wilcoxon two-group test);
^{a}p < 0.05,
^{b}p < 0.01,
^{c}p < 0.001

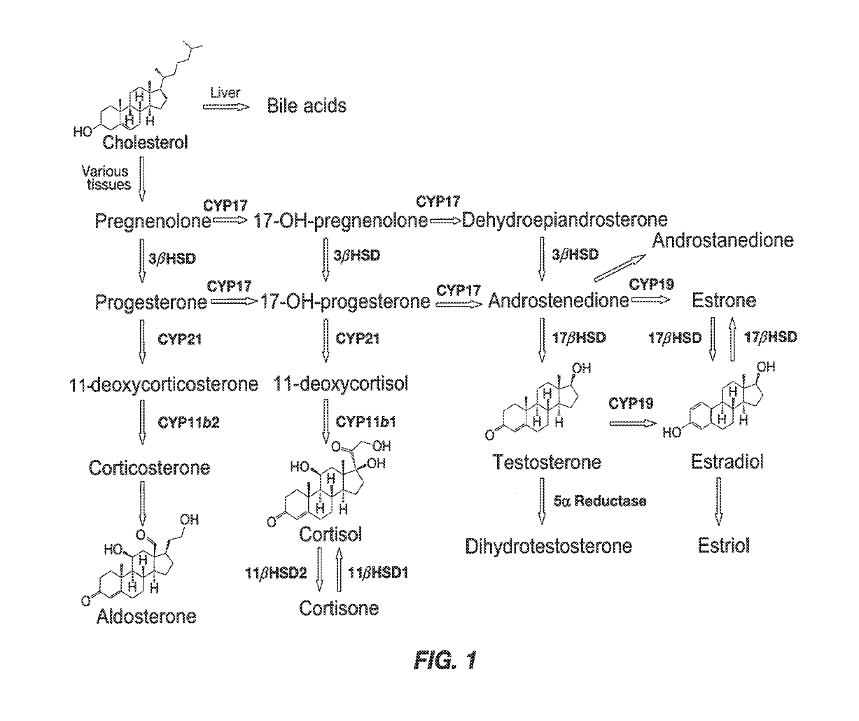

When six product/precursor ratios, illustrating enzyme activities in the pathway of steroid biosynthesis (FIG. 1) were evaluated simultaneously, the AUC reached 0.99. However, the only significant and independent ratio was 17OHPregn/Preg, p=0.021. When evaluated alone, 17OHPregn/Pregn yielded AUC=0.95, p=0.0027. The optimal cut-off value for the 17OHPregn/Pregn ratio was found to be 0.89 and yielded a sensitivity of 89% and a specificity of 90%. When E1/A4 (CYP19) and 17OHPregn/Pregn (CYP 17) were included in the same model, the AUC=0.96. However, only the 17OHPregn/Pregn ratio had an independent effect (p=0.019), suggesting the strong impact of increased CYP17 activity in FF of the PCOS patients.

In ROC analysis, the highest values of AUC were found for 17OHPregn/Pregn, A4/17OHProg, total ANDR, DHEA, A4 and the ratio of total ANDR/total ESTR, all pointing to higher activity of CYP17 and a lower activity of CYP19 in women diagnosed with PCOS as compared to women without PCOS.

The distribution of concentrations (Table 8), product/precursor ratios (Table 9) and the ROC analysis suggest higher activity of the enzyme CYP17 and a lower activity of the enzyme CYP19 (aromatase) in women diagnosed with PCOS. The results of the present study favor the hypothesis of a reduced activity/inhibition of aromatase enzyme in the ovaries of PCOS women compared with RM women. The present data also indicates a strong influence of increased CYP17 activity leading to increasing concentrations of FF androgens.

### Example III

### Analysis of Steroid Profiles in Ovarian FF Following Ovarian Stimulation in Women Undergoing IVF Treatment

### Participants

Follicular fluid was sampled from patients attending IVF treatment at Uppsala University hospital (Uppsala, Sweden). Reasons for infertility in these patients included male factor infertility, tubal factor infertility, non-ovarian endometriosis and unexplained infertility. Most currently, the treatment protocol consists of pituitary down-regulation by GnRH analog (Suprecur: Sanofi-avensis) employing the “long” protocol initiated at the mid-luteal phase (1200 micrograms/day, intranasal administration). Recombinant FSH (Puregon: Schering-Plough) was injected daily (100-450 IU/day) starting on cycle day 3 (subcutaneous injection). Dose adjustment was performed, when necessary, from cycle day 7. Human chorionic gonadotropin (hCG) (Pregnyl: Schering-Plough), 10,000 IU, was administered when one or more follicles reached a diameter of >17 mm, additional details and modifications being included in Table 10.

### Follicle Fluid Collection and Analysis

Transvaginal oocyte retrieval was performed under ultrasound guidance 36-38 hours after HCG administration. Follicles larger than 15 mm in diameter were aspirated. FF samples were kept frozen at −20° C. until analysis. The reagents and standards for follicular fluid analysis were the same as described previously in Example I. Likewise, the LC-MS/MS methods for this aspect of the invention were the same as previously described in Example I.

Thirteen subjects had a positive outcome (viable fetus by ultrasound and delivered babies) following IVF treatment, while the remaining 33 subjects had a negative outcome. Negative outcomes included failure to become pregnant (29 subjects) and spontaneous abortion following a positive pregnancy test (4 subjects). Stimulation protocols and IVF methodology did not correlate with outcome (data not shown). Table 10, below, shows information on the participants and the treatments. Table 11 shows concentrations of steroids in FF samples of women undergoing IVF treatment, and ratios of concentrations of the steroids and IVF outcome.

TABLE 10

| | | Previous | Starting | | | | Number | Urine HCG | |
|---|---|---|---|---|---|---|---|---|---|
| Sample | Age at start | IVF | Dose of | Total dose | # days with | hCG given | oocytes | (positive or | |
| # | of stimulation | attempts | FSH (IU) | FSH (IU) | FSH given | at day | retrieved | negative) | Ultrasound result |
| 6642 | 33 | 4 | 200 | 1900 | 10 | 10 | 11 | + | Viable fetus |
| 6653 | 35 | 3 | 300 | 2700 | 9 | 9 | 10 | − | No pregnancy |
| 6654 | 26 | 1 | 125 | 875 | 10 | 10 | 11 | + | Positive pregnancy |
| | | | | | | | | | test, no viable fetus |
| 6655 | 38 | 1 | 250 | 3050 | 13 | 13 | 11 | − | No pregnancy |
| 6658 | 30 | 2 | 150 | 1350 | 9 | 9 | 3 | + | Viable fetus |
| 6659 | 30 | 1 | 100 | 700 | 7 | 7 | 3 | − | No pregnancy |
| 6660 | 25 | 1 | 300 | 3600 | 12 | 12 | 12 | + | Viable fetus |
| 6661 | 30 | 2 | 250 | 3500 | 14 | 14 | 10 | − | No pregnancy |
| 6662 | 35 | 1 | 225 | 3150 | 14 | 14 | 19 | − | No pregnancy |
| 6663 | 35 | 2 | 425 | 7225 | 17 | 17 | 6 | − | No pregnancy |
| 6664 | 38 | 3 | 225 | 1575 | 7 | 7 | 5 | − | No pregnancy |
| 6643 | 39 | 4 | 450 | 5400 | 12 | 12 | 3 | − | No pregnancy |
| 6665 | 39 | 5 | 375 | 3750 | 10 | 10 | 5 | − | No pregnancy |
| 6667 | 30 | 2 | 150 | 1650 | 11 | 11 | 7 | + | Viable fetus |
| 6670 | 38 | 1 | 100 | 1350 | 12 | 12 | 2 | − | No pregnancy |
| 6671 | 38 | 2 | 250 | 2300 | 11 | 11 | 8 | − | No pregnancy |
| 6672 | 38 | 2 | 150 | 1650 | 11 | 11 | 11 | + | Positive pregnancy |
| | | | | | | | | | test, no viable fetus |
| 6673 | 36 | 3 | 300 | 3900 | 13 | 13 | 12 | + | Viable fetus |
| 6674 | 27 | 2 | 150 | 2200 | 13 | 12 | 7 | + | Viable fetus |
| 6675 | 37 | 2 | 150 | 1575 | 12 | 12 | 19 | − | No pregnancy |
| 6676 | 30 | 1 | 100 | 725 | 10 | 10 | 20 | − | No pregnancy |
| 6677 | 33 | 1 | 200 | 2800 | 14 | 14 | 4 | − | No pregnancy |
| 6644 | 31 | 2 | 150 | 2100 | 14 | 14 | 9 | − | No pregnancy |
| 6678 | 38 | 3 | 300 | 3900 | 13 | 13 | 10 | + | Viable fetus |
| 6686 | 34 | 3 | 300 | 3600 | 12 | 12 | 8 | − | No pregnancy |
| 6688 | 35 | 1 | 150 | 1800 | 12 | 12 | 11 | + | Positive pregnancy |
| | | | | | | | | | test, no viable fetus |
| 6689 | 39 | 5 | 300 | 3600 | 12 | 12 | 10 | − | No pregnancy |
| 6690 | 31 | 2 | 150 | 1800 | 13 | 12 | 11 | + | Positive pregnancy |
| | | | | | | | | | test, no viable fetus |
| 6691 | 33 | 3 | 105 | 1260 | 12 | 11 | 10 | + | Viable fetus |
| 6692 | 35 | 1 | 300 | 2700 | 9 | 9 | 2 | − | No pregnancy |
| 6693 | 39 | 1 | 200 | 1250 | 7 | 7 | 7 | − | No pregnancy |
| 6694 | 37 | 3 | 187.5 | 1875 | 10 | 10 | 21 | + | Viable fetus |
| 6695 | 38 | 2 | 300 | 3300 | 11 | 11 | 7 | + | Viable fetus |
| 6645 | 35 | 4 | 450 | 4500 | 11 | 11 | 9 | − | No pregnancy |
| 6698 | 31 | 1 | 100 | 1000 | 10 | 10 | 12 | − | No pregnancy |
| 6699 | 31 | 1 | 150 | 1650 | 11 | 11 | 6 | − | No pregnancy |
| 6700 | 33 | 1 | 200 | 2800 | 11 | 11 | 11 | + | Viable fetus |
| 6701 | 25 | 4 | 450 | 4050 | 9 | 9 | 13 | + | Viable fetus |
| 6702 | 30 | 2 | 300 | 3600 | 12 | 12 | 6 | − | No pregnancy |
| 6703 | 36 | 3 | 450 | 5400 | 12 | 12 | 6 | − | No pregnancy |
| 6704 | 39 | | 225 | 1800 | 8 | 8 | 12 | − | No pregnancy |
| 6705 | 39 | | 225 | 4275 | 12 | 12 | 5 | + | Viable fetus |
| 6646 | 28 | 1 | 250 | 2650 | 12 | 12 | 11 | − | No pregnancy |
| 6647 | 39 | 3 | 300 | 3600 | 12 | 12 | 3 | − | No pregnancy |
| 6648 | 24 | 1 | 150 | 1800 | 12 | 12 | 13 | + | Viable fetus |
| 6649 | 35 | 1 | 125 | 1250 | 14 | 14 | 6 | − | No pregnancy |
| 6650 | 31 | 3 | 300 | 3300 | 11 | 11 | 6 | − | No pregnancy |

TABLE 11
Steroid measurements (ng/mL), ratios, and outcomes for subjects undergoing IVF treatment.

| | | | | | | | | | | Hydroxy- | | | | | | |
|---|---|---|---|---|---|---|---|---|---|---|---|---|---|---|---|---|
| Sample | | 17-OH | 17-OH | | | | | | | proges- | | | | | | |
| ID | Pregn | Pregn | Preg | 11-DC | F | E | DHEA | A4 | Te | terone | A4 | E1 | E2 | E3 | Preg | Allopregn |
| 6642 | 504 | 2.79 | 714 | 2.59 | 64 | 12.40 | 1.06 | 2.20 | 0.02 | 10.85 | 0.00 | 15.60 | 114 | 2.38 | 13200 | 5.17 |
| 6643 | 591 | 15.20 | 3350 | 14.25 | 42 | 23.20 | 2.77 | 12.10 | 0.45 | 38.93 | 0.00 | 46.10 | 154 | 7.48 | 11100 | 1.64 |
| 6644 | 174 | 1.39 | 607 | 3.13 | 64 | 12.25 | 0.35 | 1.75 | 0.05 | 7.43 | 0.00 | 19.20 | 111 | 2.80 | 6720 | 3.44 |
| 6645 | 1030 | 4.36 | 1305 | 5.90 | 64 | 14.55 | 0.87 | 3.15 | 0.03 | 15.32 | 0.67 | 27.60 | 145 | 5.33 | 14300 | 4.98 |
| 6646 | 331 | 3.30 | 1125 | 4.73 | 40 | 13.65 | 1.39 | 15.25 | 0.41 | 12.78 | 0.72 | 14.50 | 113 | 3.18 | 14500 | 5.66 |
| 6647 | 582 | 2.30 | 626 | 1.99 | 32 | 9.71 | 0.39 | 1.89 | 0.09 | 6.99 | 0.55 | 14.60 | 112 | 2.00 | 14200 | 5.28 |
| 6648 | 582 | 2.97 | 955 | 3.47 | 34 | 10.59 | 0.70 | 2.45 | 0.03 | 8.63 | 0.00 | 12.70 | 106 | 2.59 | 22000 | 4.21 |
| 6649 | 319 | 3.47 | 1755 | 12.35 | 63 | 19.45 | 1.87 | 17.10 | 0.64 | 27.37 | 0.00 | 131.00 | 149 | 4.65 | 13200 | 3.02 |
| 6650 | 206 | 2.18 | 1210 | 9.01 | 47 | 11.60 | 1.14 | 34.20 | 2.08 | 21.40 | 0.00 | 46.10 | 148 | 5.81 | 6120 | 2.52 |
| 6653 | 382 | 1.95 | 460 | 2.07 | 40 | 11.90 | 1.18 | 1.52 | 0.06 | 6.39 | 0.00 | 59.90 | 90 | 2.80 | 11700 | 5.75 |
| 6654 | 498 | 3.83 | 617 | 4.01 | 57 | 19.00 | 2.78 | 37.55 | 0.58 | 11.47 | 0.84 | 18.80 | 125 | 3.49 | 5830 | 4.77 |
| 6655 | 1825 | 77.15 | 2445 | 8.51 | 45 | 15.35 | 14.90 | 6.85 | 0.14 | 32.33 | 0.00 | 42.60 | 162 | 5.81 | 11300 | 0.69 |
| 6658 | 367 | 3.46 | 885 | 3.08 | 43 | 12.55 | 0.67 | 3.61 | 0.08 | 10.14 | 0.00 | 17.70 | 110 | 3.31 | 13500 | 6.79 |
| 6659 | 665 | 3.67 | 908 | 4.23 | 70 | 11.25 | 0.74 | 2.57 | 0.06 | 12.88 | 0.00 | 21.50 | 146 | 4.49 | 10500 | 2.41 |
| 6660 | 487 | 4.67 | 1595 | 10.70 | 64 | 20.60 | 1.36 | 13.80 | 0.44 | 21.80 | 0.00 | 45.40 | 154 | 7.00 | 16300 | 3.42 |
| 6661 | 183 | 4.18 | 911 | 4.06 | 85 | 14.20 | 1.85 | 45.60 | 3.15 | 15.93 | 0.65 | 30.50 | 121 | 1.61 | 3650 | 1.55 |
| 6662 | 759 | 11.05 | 1535 | 4.26 | 22 | 7.89 | 1.29 | 3.54 | 0.03 | 18.90 | 0.00 | 20.50 | 126 | 3.52 | 12400 | 1.90 |
| 6663 | 373 | 2.54 | 1185 | 7.20 | 53 | 13.20 | [symbol] | 3.36 | 0.07 | 15.53 | 0.00 | 44.00 | 147 | 7.04 | 10600 | 5.63 |
| 6664 | 482 | 5.71 | 1615 | 8.48 | 35 | 12.10 | [symbol] | 2.82 | 0.04 | 17.72 | 0.00 | 40.80 | 146 | 7.04 | 12100 | 2.44 |
| 6665 | 27 | 1.00 | 726 | 6.63 | 72 | 7.34 | 0.28 | 7.38 | 0.37 | 10.41 | 0.00 | 11.30 | 107 | 1.85 | 6940 | 2.61 |
| 6667 | 454 | 5.99 | 991 | 2.76 | 44 | 15.50 | 1.53 | 3.17 | 0.07 | 12.93 | 0.00 | 13.80 | 109 | 2.88 | 13400 | 3.04 |
| 6670 | 753 | 16.75 | 2910 | 14.70 | 38 | 10.95 | 1.71 | 10.30 | 0.36 | 37.37 | 0.00 | 30.40 | 158 | 6.23 | 14000 | 3.15 |
| 6671 | 130 | 2.07 | 748 | 3.46 | 22 | 7.48 | 0.74 | 4.25 | 0.07 | 11.03 | 0.00 | 22.70 | 115 | 3.44 | 9130 | 3.61 |
| 6672 | 809 | 5.64 | 1115 | 6.94 | 42 | 10.35 | 0.59 | 1.98 | 0.05 | 14.78 | 0.00 | 34.70 | 146 | 7.49 | 12200 | 4.39 |
| 6673 | 441 | 3.28 | 1005 | 4.30 | 38 | 10.70 | 0.63 | 2.36 | 0.03 | 10.54 | 0.00 | 16.50 | 111 | 2.91 | 8600 | 4.02 |
| 6674 | 169 | 2.77 | 1012 | 6.95 | 70 | 13.20 | 1.86 | 22.55 | 0.85 | 16.06 | 0.00 | 36.70 | 149 | 5.78 | 4870 | 1.87 |
| 6675 | 305 | 1.85 | 1210 | 11.04 | 49 | 9.13 | 0.70 | 29.45 | 2.08 | 20.42 | 0.00 | 35.10 | 161 | 6.06 | 5970 | 3.75 |
| 6676 | 351 | 3.04 | 1080 | 7.38 | 32 | 10.75 | 0.55 | 18.60 | 0.62 | 13.59 | 0.00 | 47.20 | 141 | 5.32 | 14100 | 3.10 |
| 6677 | 171 | 4.67 | 903 | 8.99 | 47 | 11.45 | 1.64 | 147.00 | 8.23 | 12.94 | 2.77 | 19.30 | 109 | 0.48 | 3730 | 1.33 |
| 6678 | 179 | 3.28 | 852 | 5.10 | 51 | 28.05 | 3.03 | 117.50 | 4.41 | 19.20 | 0.00 | 32.40 | 123 | 2.28 | 3360 | 1.02 |
| 6686 | 572 | 2.50 | 629 | 1.89 | 42 | 7.78 | 0.48 | 1.53 | 0.01 | 6.86 | 0.00 | 6.37 | 67 | 1.73 | 11800 | 4.03 |
| 6688 | 717 | 9.74 | 1755 | 3.36 | 38 | 26.65 | 1.34 | 4.04 | 0.15 | 13.39 | 0.00 | 17.70 | 104 | 2.10 | 12700 | 2.02 |
| [symbol] | 337 | 3.67 | 1005 | 5.25 | 45 | 13.15 | 0.48 | 2.30 | 0.03 | 16.19 | [symbol] | 29.10 | 148 | 5.32 | 10000 | 1.63 |
| 6690 | 306 | 3.31 | 807 | 2.14 | 33 | 12.45 | 0.65 | 7.41 | 0.31 | 6.16 | 0.00 | 20.00 | 78 | 1.22 | 18000 | 8.02 |
| 6691 | 333 | 2.19 | 648 | 4.08 | 69 | 11.10 | 0.58 | 3.00 | 0.07 | 10.49 | 0.00 | 22.10 | 138 | 3.63 | 8810 | 3.89 |
| 6692 | 510 | 3.24 | 583 | 1.68 | 68 | 15.45 | 0.54 | 0.83 | 0.02 | 5.94 | 0.00 | 7.45 | 80 | 1.30 | 8670 | 4.16 |
| 6693 | 177 | 1.67 | 1130 | 7.83 | 44 | 13.65 | 0.62 | 7.94 | 0.23 | 15.64 | 0.00 | 39.80 | 156 | 5.50 | 7540 | 2.23 |
| 6694 HO | 331 | 1.97 | 669 | 3.60 | 50 | 11.25 | 0.57 | 3.26 | 0.05 | 9.40 | 0.00 | 22.60 | 135 | 3.67 | 8350 | 4.09 |
| 6695 HO | 202 | 1.43 | 941 | 6.47 | 58 | 19.55 | 0.84 | 46.20 | 2.09 | 18.59 | 0.72 | 31.40 | 143 | 5.46 | 5620 | 1.92 |
| 6696 HO | 406 | 4.17 | 646 | 2.06 | 29 | 10.40 | 0.71 | 1.48 | 0.03 | 6.74 | 0.43 | 5.92 | 81 | 1.76 | 10500 | 6.17 |
| 6699 HO | 351 | 4.72 | 1415 | 9.54 | 57 | 14.10 | 0.61 | 7.83 | 0.07 | 17.13 | 0.00 | 26.90 | 144 | 4.81 | 8730 | 2.51 |
| 6700 HO | 604 | 3.07 | 1610 | 6.54 | 75 | 15.10 | 0.63 | 3.28 | 0.04 | 20.77 | 0.00 | 23.20 | 144 | 4.33 | 7770 | 4.47 |
| 6701 HO | 333 | 3.06 | 1099 | 6.90 | 47 | 12.45 | 1.66 | 9.63 | 0.23 | 18.83 | 0.00 | 40.60 | 152 | 6.71 | 7910 | 1.17 |
| 6702 HO | 453 | 2.53 | 1080 | 5.40 | 39 | 15.35 | 0.68 | 1.63 | 0.02 | 11.15 | 0.00 | 15.30 | 121 | 2.63 | 12900 | 1.83 |
| 6703 HO | [symbol] | 10.80 | 1845 | 8.66 | 38 | 11.10 | 1.28 | 4.67 | 0.11 | 26.18 | 0.00 | 40.50 | 154 | 5.67 | 10200 | 2.85 |
| 6704 HO | 355 | 2.31 | 620 | 2.44 | 52 | 10.60 | 0.81 | 1.97 | 0.03 | 7.86 | 0.32 | 11.00 | 96 | 1.85 | 6340 | 4.07 |

| | | | Total | | | | | | Ratio | Ratio | | | |
|---|---|---|---|---|---|---|---|---|---|---|---|---|---|
| Sample | Total | Total | ANDR (with | Total | Total | Total | Ratio | Ratio | 17OH-Pregn/ | 17OH Preg/ | Ratio | Ratio | Ratio |
| ID | ESTR | ANDR | isomer) | OC | [symbol] | [symbol] | Preg/E3 | E/F | Pregn | Pregn | E2/E1 | E3/E2 | E3/E1 |
| 6642 | 132 | 3.29 | 14 | 75.90 | 13914 | [symbol] | 5546 | 0.20 | 0.006 | 1.42 | 7.31 | 0.02 | 0.15 |
| 6643 | 20 [symbol] | 15.32 | 54 | 64.80 | 14450 | 606 | 1484 | 0.56 | 0.026 | 5.67 | 3.34 | 0.05 | 0.16 |
| 6644 | 133 | 2.14 | 10 | 75.85 | 7527 | 175 | 2400 | 0.19 | 0.008 | 4.64 | 5.78 | 0.03 | 0.15 |
| 6645 | 178 | 4.72 | 20 | 78.40 | 15605 | 1034 | 2683 | 0.23 | 0.004 | 1.27 | 5.25 | 0.04 | 0.19 |
| 6646 | 131 | 17.76 | 31 | 53.35 | 15625 | 334 | 4560 | 0.34 | 0.010 | 3.40 | 7.79 | 0.03 | 0.22 |
| 6647 | 129 | 2.92 | 10 | 42.11 | 14825 | 584 | 7100 | 0.30 | 0.004 | 1.08 | [symbol] | 0.02 | 0.14 |
| 6648 | 121 | 3.18 | 12 | 44.09 | 22965 | 584 | 8494 | 0.32 | 0.005 | 1.66 | 8.35 | 0.02 | 0.20 |
| 6649 | 285 | 19.61 | 47 | 102.80 | 14955 | 322 | [symbol] | 0.23 | 0.011 | 5.50 | [symbol] | 0.03 | 0.04 |
| 6650 | 200 | 37.41 | 59 | 58.50 | 7330 | 208 | 1053 | 0.25 | 0.011 | 5.87 | 3.21 | 0.04 | 0.13 |
| 6653 | 153 | 2.76 | 9 | 52.25 | 12160 | 354 | 4179 | 0.29 | 0.005 | 1.20 | 1.51 | 0.03 | 0.05 |
| 6654 | 147 | 41.75 | 53 | 76.45 | 6447 | [symbol] | 1670 | 0.33 | 0.006 | 1.24 | [symbol] | 0.03 | 0.19 |
| 6655 | 210 | 21.89 | 54 | 60.15 | 13745 | 1902 | 1945 | 0.34 | 0.042 | 1.34 | [symbol] | 0.04 | 0.14 |
| 6658 | 131 | 4.33 | 14 | 55.95 | 14595 | 370 | 4109 | 0.29 | 0.009 | 2.71 | 4.21 | 0.03 | 0.19 |
| 6659 | 172 | 3.36 | 16 | 81.10 | 11408 | 668 | 2339 | 0.16 | 0.006 | 1.37 | 4.79 | 0.03 | 0.21 |
| 6660 | 208 | 15.60 | 37 | 85.00 | 17895 | 492 | 2329 | 0.32 | 0.010 | 3.28 | 3.59 | 0.05 | 0.15 |
| 6661 | 153 | 51.25 | 67 | 98.90 | 4561 | 187 | 2267 | 0.17 | 0.023 | 4.98 | 3.97 | 0.01 | 0.05 |
| 6662 | 150 | 4.86 | 24 | 29.29 | 13935 | 770 | 3523 | 0.37 | 0.015 | 2.02 | 6.15 | 0.03 | 0.17 |
| 6663 | 198 | 4.31 | 20 | 56.05 | 11785 | [symbol] | 1506 | 0.25 | 0.007 | 3.18 | 3.34 | 0.05 | 0.16 |
| 6664 | 194 | 3.66 | 21 | 46.75 | 13715 | 488 | 1719 | 0.35 | 0.012 | 3.35 | [symbol] | 0.05 | 0.17 |
| 6665 | 120 | 8.02 | 18 | 75.94 | 7668 | 26 | 3751 | 0.10 | 0.037 | 26.82 | 9.47 | 0.02 | 0.16 |
| 6667 | 126 | 4.76 | 18 | 59.55 | 14391 | 460 | 4653 | 0.35 | 0.013 | 2.18 | 7.90 | 0.03 | 0.21 |
| 6670 | 195 | 12.36 | 50 | 48.75 | 16910 | 769 | 2247 | 0.29 | 0.022 | 3.87 | 5.20 | 0.04 | 0.20 |
| 6671 | 141 | 5.06 | 16 | 29.53 | 9878 | 132 | 2654 | 0.34 | 0.016 | 5.77 | 5.07 | 0.03 | 0.15 |
| 6672 | 188 | 2.83 | 17 | 52.70 | 13315 | 614 | [symbol] | 0.24 | [symbol] | [symbol] | 4.21 | 0.05 | 0.22 |
| 6673 | 130 | 3.01 | 14 | 48.35 | 9605 | 444 | 2955 | 0.28 | 0.007 | 2.28 | 6.73 | 0.03 | 0.16 |
| 6674 | 191 | 25.25 | 41 | 83.55 | 5882 | 172 | [symbol] | 0.19 | [symbol] | 5.99 | 4.06 | 0.04 | 0.16 |
| 6675 | 202 | 32.23 | 53 | 57.98 | 7180 | 307 | [symbol] | [symbol] | [symbol] | 3.97 | 4.59 | 0.04 | 0.17 |
| 6676 | 194 | 19.77 | 33 | 42.55 | 15190 | 354 | [symbol] | 0.34 | [symbol] | 3.11 | 2.99 | 0.04 | 0.11 |
| 6677 | 129 | 157.64 | 171 | 56.60 | 4633 | 176 | 7659 | 0.24 | 0.027 | 5.28 | 5.65 | [symbol] | 0.03 |
| 6678 | 158 | 124.94 | 144 | 68.65 | 4212 | 182 | 1474 | 0.46 | 0.018 | 4.77 | 3.60 | 0.02 | 0.07 |
| 6686 | 95 | 2.02 | 9 | 50.13 | 12429 | 574 | 6821 | [symbol] | 0.004 | 1.10 | 13.59 | 0.02 | 0.27 |
| 6688 | 124 | 5.52 | 19 | 64.65 | 14455 | 726 | 6048 | 0.70 | 0.014 | 2.45 | [symbol] | [symbol] | 0.12 |
| [symbol] | 182 | 3.42 | 20 | 58.60 | 11005 | 341 | 1880 | 0.29 | 0.011 | 2.98 | [symbol] | 0.04 | 0.18 |
| 6690 | 99 | [symbol] .37 | 15 | 45.70 | 16807 | 309 | 13115 | 0.37 | 0.011 | 2.64 | [symbol] | 0.02 | 0.06 |
| 6691 | 164 | 3.65 | 14 | 69.85 | 9458 | [symbol] | [symbol] | 0.19 | 0.007 | 1.95 | [symbol] | [symbol] | 0.17 |
| 6692 | 88 | 1.39 | 7 | 63.60 | 9253 | 513 | [symbol] | 0.23 | 0.008 | 1.14 | 10.68 | 0.02 | 0.17 |
| 6693 | 201 | [symbol] .80 | 24 | 57.70 | 8670 | 179 | 1371 | 0.31 | 0.009 | [symbol] | 3.92 | 0.04 | 0.14 |
| 6694 HO | 161 | 3.86 | 13 | 68.90 | 9019 | 333 | 2275 | 0.20 | 0.006 | 2.02 | 5.97 | [symbol] | 0.16 |
| 6695 HO | 180 | 49.84 | 68 | 77.80 | 6561 | 203 | 1029 | 0.34 | 0.007 | 4.65 | 4.55 | 0.04 | 0.17 |
| 6696 HO | [symbol] | 2.65 | 9 | 39.60 | 11348 | 412 | [symbol] | 0.36 | [symbol] | 2.08 | [symbol] | 0.02 | 0.30 |
| 6699 HO | 178 | [symbol] .51 | 26 | 71.30 | 10145 | 355 | 1815 | 0.25 | 0.013 | 4.04 | [symbol] | 0.03 | 0.17 |
| 6700 HO | 172 | 3.94 | 25 | 89.60 | 8380 | 607 | 1794 | 0.20 | 0.005 | 2.67 | [symbol] | 0.03 | 0.19 |
| 6701 HO | 200 | 11.51 | 30 | 59.55 | 9009 | 336 | 1179 | 0.26 | [symbol] | 3.30 | 3.73 | 0.04 | 0.16 |
| 6702 HO | 139 | 2.31 | 13 | 54.80 | 13960 | 456 | 4905 | 0.39 | [symbol] | 2.34 | 7.91 | 0.02 | 0.17 |
| 6703 HO | 203 | 6.00 | 32 | 48.85 | 12045 | [symbol] | 1176 | 0.29 | 0.012 | 1.97 | 3.80 | [symbol] | 0.21 |
| 6704 HO | 109 | 3.12 | 11 | 62.40 | 6960 | 357 | 3427 | 0.20 | 0.007 | 1.75 | [symbol] | 0.02 | 0.17 |

| | Ratio Total | Ratio | | Ratio Total | Ratio Total | Ratio Total | Ratio | | |
|---|---|---|---|---|---|---|---|---|---|
| Sample | ANDR/Total | Preg/17OH | Ratio | progestines/ | progestines/ | progestines/ | Pregn/ | Ratio | |
| ID | ESTR | [symbol] | [symbol] /E2 | total ESTR | total OC | total ANDR | Allopregn | A4/11OC | [symbol] |
| 6642 | 0.02 | 18.49 | 115.79 | 105.43 | 183 | 4226 | 97.49 | [symbol] | Viable fetus |
| 6643 | 0.07 | 3.31 | [symbol] | 69.61 | 223 | 943 | 380.37 | [symbol] | No pregnancy |
| 6644 | 0.02 | [symbol] | [symbol] | 56.59 | [symbol] | 3510 | 50.58 | 0.56 | No pregnancy |
| 6645 | 0.03 | 10.96 | 96.62 | 87.70 | 199 | 3306 | 206.83 | 0.53 | No pregnancy |
| 6646 | 0.14 | 12.69 | [symbol] | 119.57 | [symbol] | [symbol] | 58.48 | 3.23 | No pregnancy |
| 6647 | 0.02 | 22.68 | 126.79 | 115.29 | 352 | 5076 | 110.13 | 0.95 | No pregnancy |
| 6648 | 0.03 | 22.80 | [symbol] | 189.34 | 521 | [symbol] | [symbol] | 0.71 | Viable fetus |
| 6649 | 0.07 | 7.52 | 88.59 | 52.54 | 145 | 763 | [symbol] | 1.35 | No pregnancy |
| 6650 | 0.19 | 5.06 | 41.35 | 36.67 | 125 | 196 | 81.75 | [symbol] | No pregnancy |
| 6653 | 0.02 | [symbol] | 129.42 | 79.43 | 233 | 4414 | 66.43 | 0.73 | No pregnancy |
| 6654 | 0.28 | 9.46 | 46.64 | 43.77 | 84 | 154 | 104.40 | [symbol] | Lost pregnancy |
| 6655 | 0.10 | 4.62 | 69.75 | 65.32 | 229 | 628 | [symbol] | [symbol] | No pregnancy |
| 6658 | 0.03 | 13.68 | 123.64 | 111.40 | 261 | 3372 | 53.96 | 1.17 | Viable fetus |
| 6659 | 0.02 | [symbol] | 71.92 | [symbol] | 141 | [symbol] | 275.73 | 0.61 | No pregnancy |
| 6660 | 0.06 | 10.22 | 105.84 | [symbol] | 211 | 1147 | 142.40 | 1.29 | Viable fetus |
| 6661 | 0.33 | 4.01 | 30.17 | 29.79 | 46 | 89 | 118.06 | 11.25 | No pregnancy |
| 6662 | 0.03 | [symbol] | 96.41 | 92.89 | 474 | 2870 | 399.47 | 0.83 | No pregnancy |
| 6663 | 0.02 | 8.95 | 72.11 | 59.51 | 175 | 2733 | [symbol] | 0.47 | No pregnancy |
| 6664 | [symbol] | 7.49 | [symbol] | 70.75 | [symbol] | 3744 | 197.54 | 0.33 | No pregnancy |
| 6665 | 0.07 | 9.57 | 64.86 | [symbol] | 97 | [symbol] | 10.36 | 1.11 | No pregnancy |
| 6667 | 0.04 | 13.53 | 122.94 | 114.50 | [symbol] | [symbol] | 149.34 | 1.15 | Viable fetus |
| 6670 | 0.06 | 4.81 | [symbol] | [symbol] | 347 | 1368 | 238.89 | [symbol] | No pregnancy |
| 6671 | 0.04 | 12.21 | 79.39 | 69.96 | 334 | 1953 | 35.87 | 1.23 | No pregnancy |
| 6672 | 0.01 | 10.94 | [symbol] | 70.75 | 253 | 5059 | 135.61 | 0.29 | Lost pregnancy |
| 6673 | 0.02 | 8.56 | 77.48 | 73.65 | 199 | [symbol] | 109.58 | 0.55 | Viable fetus |
| 6674 | 0.13 | [symbol] | 32.68 | 30.72 | 70 | 233 | [symbol] | 3.24 | Viable fetus |
| 6675 | 0.16 | 4.93 | 37.06 | 35.52 | 124 | 223 | 81.33 | 2.87 | No pregnancy |
| 6676 | 0.10 | 12.94 | 100.00 | 78.49 | 357 | 768 | 113.23 | 2.52 | No pregnancy |
| 6677 | 1.22 | 4.13 | 34.22 | 35.97 | 79 | 29 | 128.57 | 18.35 | No pregnancy |
| 6678 | 0.79 | 3.95 | 27.32 | 26.71 | 48 | 34 | [symbol] | 23.04 | Viable fetus |
| 6686 | 0.02 | 15.77 | 136.25 | 131.24 | 248 | [symbol] | 141.94 | 0.61 | No pregnancy |
| 6688 | 0.04 | 7.24 | 122.12 | 116.76 | 224 | 2619 | 354.70 | 1.20 | Lost pregnancy |
| [symbol] | [symbol] | [symbol] | 67.57 | 60.33 | 1854 | 3221 | 206.75 | 0.44 | No pregnancy |
| 6690 | [symbol] | [symbol] | 205.13 | [symbol] | [symbol] | 2009 | 38.09 | 3.46 | Lost pregnancy |
| 6691 | 0.02 | [symbol] | 63.64 | 57.70 | 135 | 2591 | 85.48 | 0.74 | Viable fetus |
| 6692 | 0.02 | 14.87 | 106.92 | 104.73 | 111 | [symbol] | 122.60 | 0.50 | No pregnancy |
| 6693 | 0.04 | [symbol] | 48.33 | 43.07 | 150 | [symbol] | [symbol] | 1.01 | No pregnancy |
| 6694 HO | 0.02 | 12.49 | 61.85 | 55.92 | 131 | 2324 | 80.93 | 0.56 | Viable fetus |
| 6695 HO | 0.28 | 5.97 | 39.30 | 36.48 | [symbol] | 132 | 105.21 | 7.14 | Viable fetus |
| 6696 HO | 0.03 | 12.39 | 130.43 | 128.69 | 267 | [symbol] | [symbol] | 0.72 | No pregnancy |
| 6699 HO | 0.05 | 6.17 | 60.63 | 57.09 | 142 | [symbol] | 134.29 | 0.62 | No pregnancy |
| 6700 HO | 0.02 | 4.63 | 53.96 | 54.68 | 105 | 2380 | 135.12 | 0.50 | Viable fetus |
| 6701 HO | 0.06 | 7.20 | 52.04 | 45.15 | [symbol] | 782 | 264.62 | 1.38 | Viable fetus |
| 6702 HO | 0.02 | 12.17 | 106.81 | 100.48 | [symbol] | [symbol] | 247.54 | 0.30 | No pregnancy |
| 6703 HO | 0.03 | 5.53 | 66.23 | 59.29 | 247 | 1988 | [symbol] | 0.53 | No pregnancy |
| 6704 HO | 0.03 | 10.23 | 66.25 | 64.12 | 112 | 2228 | 87.10 | 0.81 | No pregnancy |

[symbol]

indicates data missing or illegible when filed

### Median Values and Percentiles

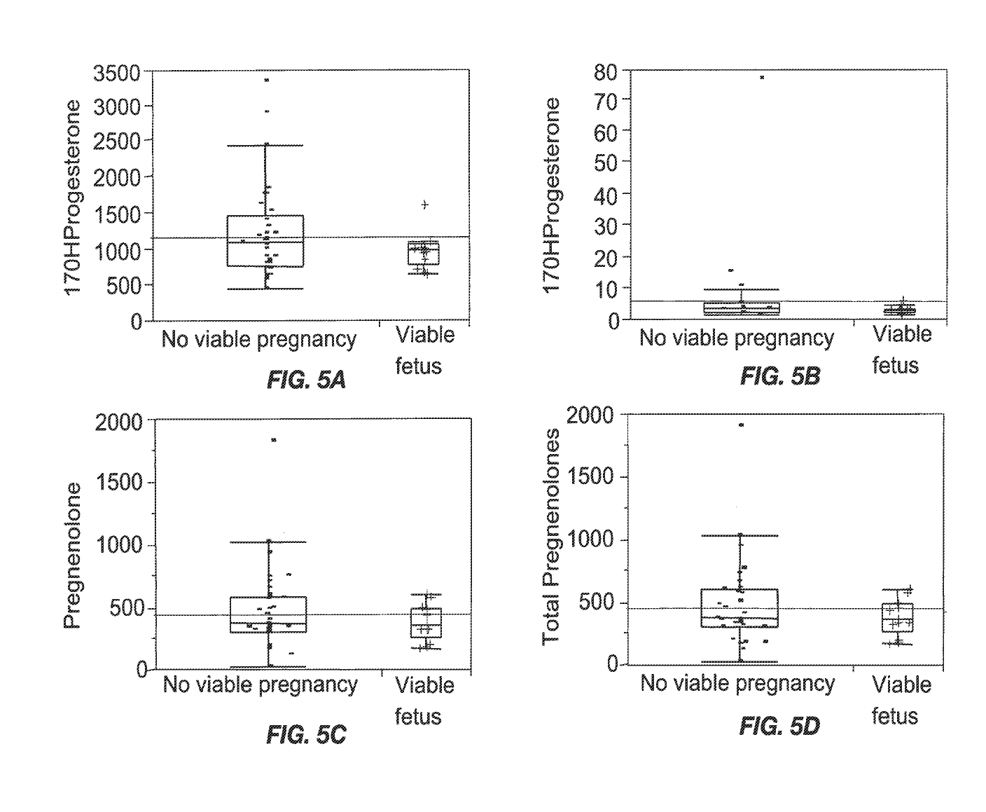

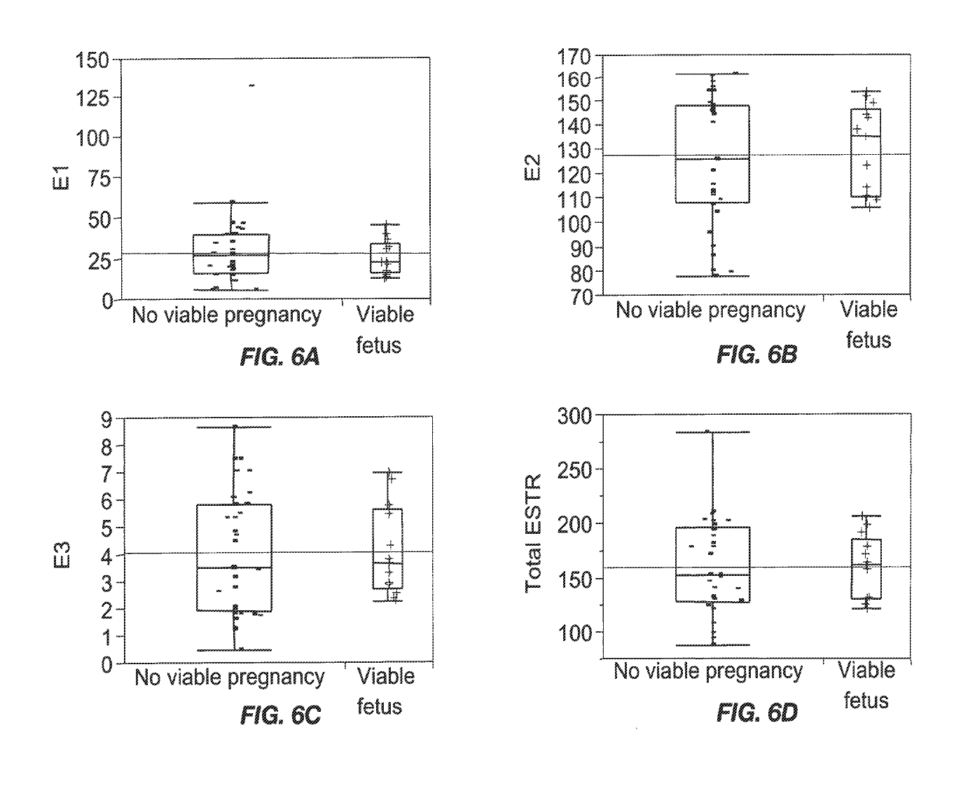

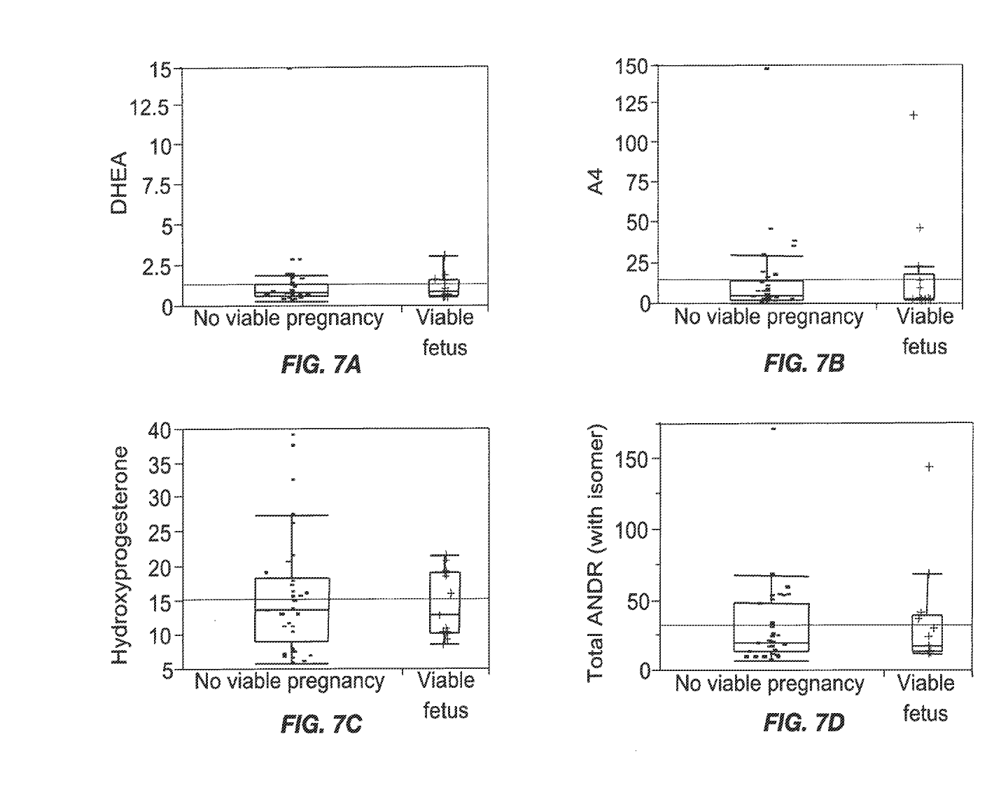

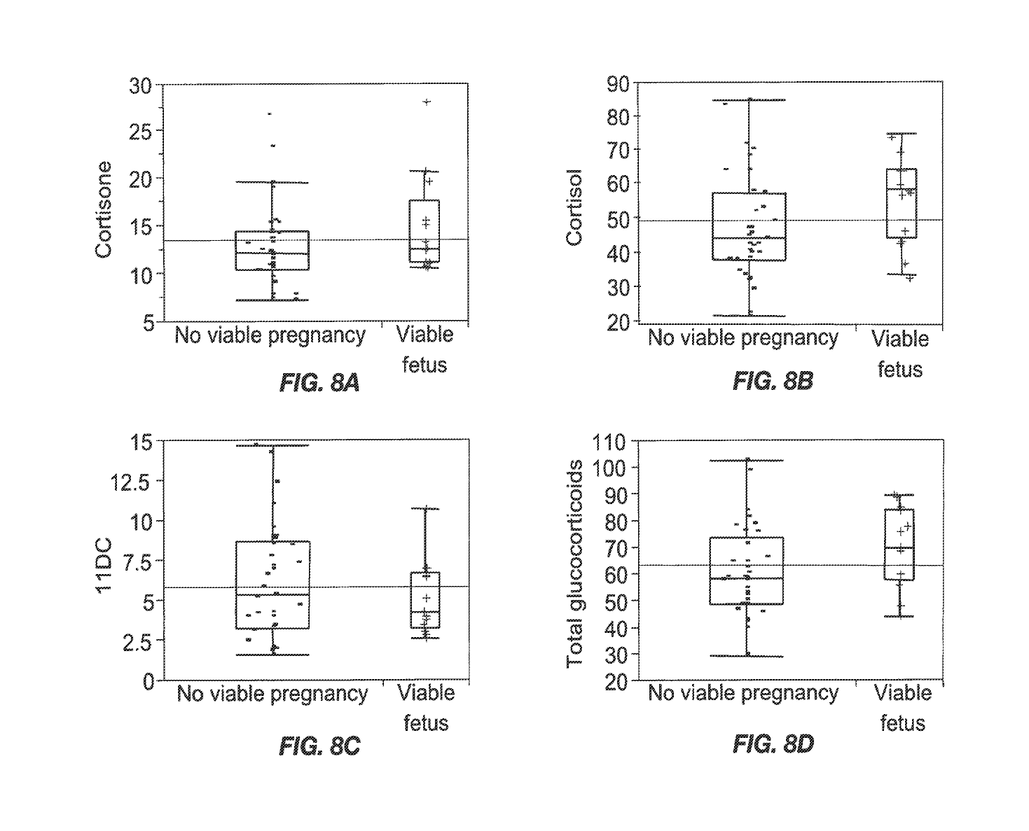

FIGS. 5-8 show graphical representations of observed values for steroid concentrations associated with both positive and negative IVF outcomes. Median values for concentrations of steroids and ratios were grouped for the subjects based on the outcomes (viable pregnancy vs. no viable pregnancy), along with the central 90th percentile of these values, as shown in Table 12, below.

TABLE 12
Median 5^{th} and 95^{th} percentile of concentrations (ratios of concentrations) of steroids
measured in FF in groups with viable pregnancy and no viable pregnancy.

| | Viable pregnancy | | | No viable pregnancy | | | % Difference | |
|---|---|---|---|---|---|---|---|---|
| Analyte (concentrations in ng/mL) | Median | 5^{th} | 95th | Median | 5th | 95th | 5th | 95th |
| 17OHProgesterone | 990.5 | 660.3 | 1601 | 1090 | 603.1 | 2631 | −9% | 64% |
| Hydroxyprogesterone | 12.9 | 9.1 | 21.1 | 13.6 | 6.3 | 34.3 | −31% | 63% |
| 11deoxycortisol | 4.30 | 2.69 | 8.46 | 5.40 | 1.95 | 13.11 | −28% | 55% |
| Total estrogens | 161 | 124 | 202 | 153 | 92 | 209 | −26% | 3% |
| Pregnenolone | 367 | 175 | 591 | 382 | 154 | 975 | −12% | 65% |
| Estrone | 22.60 | 13.36 | 42.64 | 27.60 | 7.02 | 52.28 | −47% | 23% |
| Estradiol | 135 | 108 | 153 | 126 | 80 | 159 | −26% | 4% |
| Estriol | 3.67 | 2.34 | 6.83 | 3.52 | 1.27 | 7.48 | −46% | 10% |
| Androstenedione | 3.28 | 2.29 | 74.72 | 4.25 | 1.50 | 40.77 | −35% | −45% |
| 17OHPregnenolone | 3.06 | 1.75 | 5.20 | 3.57 | 1.77 | 15.90 | 1% | 206% |
| Total androgens | 4.3 | 3.1 | 79.9 | 5.5 | 2.1 | 45.5 | −33% | −43% |
| DHEA | 0.84 | 0.58 | 2.32 | 0.81 | 0.37 | 2.77 | −35% | 19% |
| Cortisol | 58.3 | 36.0 | 72.0 | 44.1 | 26.3 | 76.3 | −27% | 6% |
| Cortisone | 12.55 | 10.66 | 23.58 | 12.10 | 7.66 | 20.95 | −28% | −11% |
| Total glucocorticoids | 69.9 | 46.6 | 89.0 | 58.5 | 35.6 | 89.7 | −24% | 1% |
| Progesterone | 8600 | 4266 | 18580 | 10600 | 4990 | 14380 | 17% | −23% |
| Total pregnenolones | 370 | 178 | 594 | 384 | 158 | 983 | −11% | 66% |
| Ratio 17OH-Pregnenolone/ | 0.007 | 0.005 | 0.017 | 0.010 | 0.004 | 0.031 | −15% | 82% |
| Pregnenolone | | | | | | | | |
| Ratio 17OH Progesterone/ | 2.67 | 1.56 | 5.26 | 2.98 | 1.13 | 6.08 | −28% | 16% |
| Pregnenolone | | | | | | | | |
| Ratio estradiol/estrone | 6.21 | 3.59 | 8.08 | 5.09 | 2.40 | 11.85 | −33% | 47% |
| Ratio estriol/estradiol | 0.028 | 0.020 | 0.045 | 0.031 | 0.015 | 0.050 | −26% | 11% |
| Ratio estriol/estrone | 0.173 | 0.120 | 0.206 | 0.166 | 0.042 | 0.240 | −65% | 17% |
| Ratio Pregnenolone/ | 109.6 | 70.1 | 218.8 | 118.1 | 37.2 | 376.0 | −47% | 72% |
| Allopregnenolone | | | | | | | | |
| Ratio A4/11deoxycortisol | 1.15 | 0.53 | 13.50 | 0.82 | 0.32 | 10.12 | −40% | −25% |

The percent difference between the 5th percentile and 95th percentile values associated with each group were also determined. This analysis reveals differences in the distribution of the values for specific analytes between the groups. In comparison to the group with viable pregnancies, negative outcomes were associated with an altered distribution of steroid concentration. Steroids for which 95th percentile values were markedly elevated by approximately 50% or more in the group with no viable pregnancy, compared with those with viable pregnancy, were 17-OH progesterone, 17-OH pregnenolone, pregnenolone and total pregnenolones (pregnenolone and 17-OH pregnenolone), indicating that higher concentrations of these steroids in FF may serve as markers predictive of a decreased probability of viable pregnancy.

Analytes for which 5th percentile values were decreased by 20% or more in the group with no viable pregnancy, compared with those with viable pregnancy, were E1, E2, E3, DHEA, A4, cortisol, cortisone, total estrogens (estrone, estradiol and estrone), and total glucocorticoids (cortisol, cortisone). The 95th percentile values for A4 and total androgens (A4, DHEA, and Te) were also markedly decreased in this group. Thus, lower concentrations of one or more of these steroids in FF may also be an indicator of a decreased likelihood of viable pregnancy. For some analytes, particularly hydroxyprogesterone (a chromatographic peak which chilled at relative retention times of 0.89 relative to progesterone and 1.15 relative to 17-hydroxyprogesterone and possessing the same characteristic mass transitions as progesterone and 17-hydroxyprogesterone), 11DC, estrone, pregnenolone, androstenedione, total ANDR, as well as the ratio 17OH-pregnenolone; pregnenolone and the ratio estradiol/estrone, it appears that both elevated and lowered values are associated with a decreased likelihood of viable pregnancy.

To determine the frequency of the steroid levels occurring outside of the distribution of the values observed in the group with no viable pregnancies compared to the viable pregnancy group, data were evaluated as follows: The minimum and maximum observed values for concentration of each steroid or ratios of concentrations of steroids in the group with viable pregnancies were determined, and the number of samples from the group with no viable pregnancy which fell outside of this range, were calculated, as shown in Table 13, below.

TABLE 13
Maximum of minimum values of concentrations of steroids
(ng/mL) or ratios of concentrations of steroids observed in group of patients with
viable pregnancy and number of samples with values of the markers above and
below the distribution observed in the group of patients with no viable pregnancy.

| | | | No viable pregnancy | |
|---|---|---|---|---|
| | | | N = 33 | |
| | | | No. of samples | No. of samples |
| | Viable Pregnancy | | in group above | in group below |
| | N = 13 | | the distribution | the distribution |
| | Maximum | Minimum | seen in viable | seen in viable |
| Analyte in ng/mL | observed value | observed value | pregnancy | pregnancy |
| 17OHProgesterone | 1610 | 648 | 8 | 0 |
| 11DC | 10.70 | 2.59 | 4 | 6 |
| Pregnenolone | 604 | 169 | 8 | 1 |
| 17OHPregnenolone | 5.99 | 1.43 | 6 | 0 |
| E1 | 45.40 | 12.70 | 4 | 5 |
| E2 | 154 | 106 | 0 | 6 |
| E3 | 7.00 | 2.28 | 0 | 8 |
| A4 | 117 | 2.2 | 0 | 8 |
| Hydroxyprogesterone | 8.63 | 21.60 | 5 | 8 |
| Cortisone | 28.05 | 10.6 | 0 | 6 |
| Cortisol | 74.5 | 33.5 | 2 | 2 |
| DHEA | 3.03 | 0.57 | 1 | 5 |
| Total estrogens | 206.4 | 121.3 | 3 | 6 |
| Total androgens | 125 | 3.01 | 1 | 7 |
| Total glucocorticoids | 89.6 | 44.1 | 2 | 4 |
| Total pregnenolones | 607.1 | 171.8 | 8 | 2 |
| (pregnenolone + 17OH- | | | | |
| pregnenolone) | | | | |
| Ratio 17OH-Pregnenolone/Pregnenolone | 0.02 | 0.01 | 6 | 8 |
| Ratio 17OH Progesterone/Pregnenolone | 5.99 | 1.42 | 2 | 8 |
| Ratio E2/E1 | 8.35 | 3.39 | 5 | 4 |
| Ratio E3/E2 | 0.05 | 0.02 | 1 | 2 |
| Ratio E3/E1 | 0.21 | 0.07 | 4 | 5 |
| Ratio | 284.62 | 53.98 | 5 | 4 |
| Pregnenolone/Allopregnenolone | | | | |
| Ratio A4/11deoxycortisol | 23.04 | .05 | 0 | 5 |

Values from the group with no viable pregnancy which were above the maximum values seen in the group with viable pregnancy were designated “out of range high”, and those which were below the minimum values were designated “out of range low.” A chi-square test was performed to determine statistical significance of the findings.

The results of this analysis suggest that elevated concentrations of 17-OH progesterone, pregnenolone, 17-OH pregnenolone, and total pregnenolones in FF are significantly less likely to be associated with a viable pregnancy, as illustrated in Table 14, below.

TABLE 14
Percent of samples in group with no viable pregnancy, which
have concentration or ratio of concentrations of steroids above the
distribution in group with viable pregnancy and p-values for
significance of the observed differences between the groups
(Chi-Square test).

| | % of samples out | |
|---|---|---|
| | of range high in | |
| | group with no | |
| Analyte | viable pregnancy | p-value |
| 17OHProgesterone | 24% | 0.0003 |
| Pregnenolone | 24% | 0.0002 |
| Total pregnenolones | 24% | 0.0002 |
| 17OHPregnenolone | 18% | 0.0006 |
| Hydroxyprogesterone | 18% | 0.0014 |
| 11DC | 12% | 0.0032 |
| Estrone | 12% | 0.0032 |
| Estradiol | 9% | 0.0087 |
| Ratio 17OH-Pregnenolone/Pregnenolone | 18% | 0.0006 |
| Ratio E2/E1 | 15% | 0.0013 |
| Ratio Pregnenolone/Allopregn | 15% | 0.0014 |
| Ratio E3/E1 | 12% | 0.0031 |

Lower concentrations of E2, E3, A4, hydroxyprogesterone, 17-OHProg, 11-DC, E and total androgens and total estrogens in FF are also significantly less likely to be associated with a viable pregnancy, as suggested in Table 15, below. In addition, elevated ratios of 17-OH pregnenolone/pregnenolone and a lowered ratio of 17-OH progesterone/pregnenolone also appear to be indicative of a decreased likelihood of viable pregnancy.

The invention thus provides analytical means for determining the viability of oocytes for IVF based on analyzing follicular fluid samples and determining steroid profiles therefrom. The invention also provides means for determining which oocytes are unlikely to produce favorable IVF outcomes, thereby enabling the determination of the usefulness of such oocytes for stem cell protocols.

TABLE 15
Percent of samples in the group of patients with no viable
pregnancy with concentration or ratio of concentrations of steroids below
the distribution in the group with viable pregnancy and p-values for
significance of the observed differences between the groups
(Chi-square test).

| | % of samples out of | |
|---|---|---|
| | range low high in | |
| | group with no viable | |
| Analyte | pregnancy | p-value |
| Estriol | 30% | 0.0001 |
| Androstenedione | 24% | 0.0002 |
| Hydroxyprogesterone | 24% | 0.0002 |
| Total androgens | 21% | 0.0003 |
| Estradiol | 18% | 0.0006 |
| 17OHProgesterone | 18% | 0.0006 |
| 11DC | 18% | 0.0006 |
| Total estrogens | 18% | 0.0006 |
| Cortisone | 18% | 0.0006 |
| DHEA | 15% | 0.0013 |
| Estrone | 15% | 0.0014 |
| Total glucocorticoids | 12% | 0.0032 |
| Ratio 17OH Progesterone/Pregnenolone | 24% | 0.0002 |
| Ratio E3/E1 | 15% | 0.0013 |
| Ratio A4/11deoxycortisol | 15% | 0.0014 |
| Ratio E2/E1 | 12% | 0.0032 |
| Ratio Pregnenolone/Allopregn | 12% | 0.0032 |

Association of Steroid Profiles with IVF Outcome

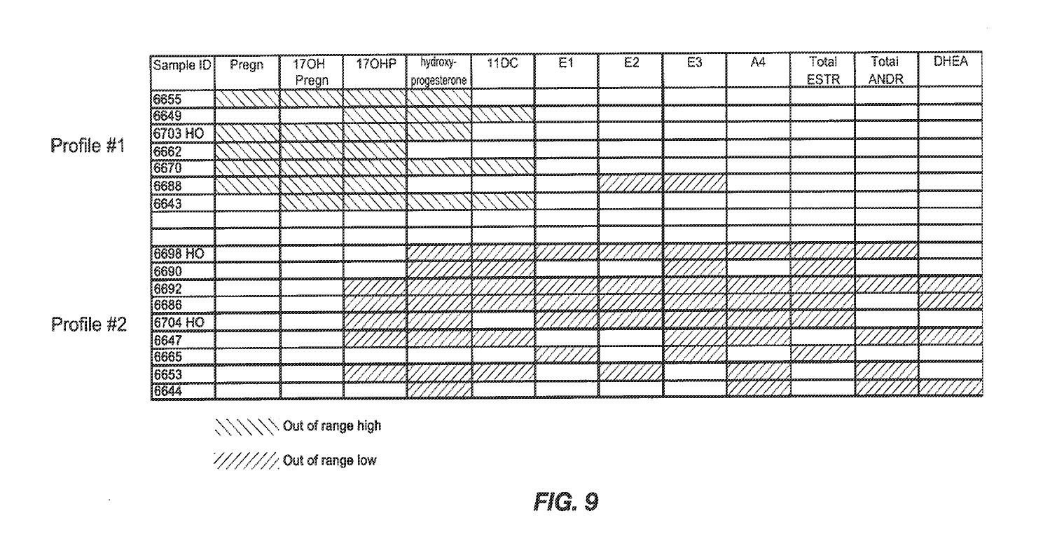

Several distinct profiles of steroid distribution in FF were observed within the group of samples from women who did not become pregnant, as shown in FIG. 9. One group is characterized by an elevated concentration of Pregn and its immediate metabolites, 17OHPreg and 17OHP (Profile 1). This profile appears to indicate an enhanced rate of steroidogenesis coupled with a deficiency in the activity of the enzymes required for biosynthesis of sex steroids. Subjects who exhibited higher concentrations of pregnenolone and its metabolites in FF were also likely to have elevated concentration of 11-DC. This profile is characterized by lower activity of enzymes CYP11, CYP17, 17βHSD, CYP19, and 3βHSD, as shown in Table 15, below. Another distinct steroid profile observed in FF of women who did not become pregnant was associated with reduced concentrations of the progestines, sex steroids 11-DC and E (Profile 2). This profile is characterized by lower activity of the enzymes CYP17, 3βHSD, CYP21, increased activity of the enzymes CYP11 and CYP19. Ratios of concentrations of the steroids which indicate these changes in enzyme activities are shown in Table 16.

TABLE 16
Median values of the ratios of concentrations of steroid
product/precursors of the pathway used as markers of enzyme activities in
women with viable pregnancy and no pregnancy (profile types 1 and 2).

| | Steroid product/precursor | | | |
|---|---|---|---|---|
| Enzyme | concentration ratios | Viable pregnancy | Profile 1 | Profile 2 |
| 3βHSD | 17OHProg/17OHPregn | 2.67 | 2.45 | 1.75 |
| CYP21 | 11DC/17OHProg | 0.0057 | 0.0043 | 0.0032^{a} |
| CYP11 | Cortisol/11DC | 11.40 | 5.02^{b} | 19.54^{b} |
| 11βHSD type 1 and 2 | Cortisone/Cortisol | 0.284 | 0.343^{a} | 0.227 |
| CYP17 | DHEA/17OHPregn | 0.29 | 0.14^{b} | 0.19^{a} |
| CYP17 | A4/17OHProg | 0.0046 | 0.0028 | 0.0030 |
| CYP17 | 17OHPregn/Pregn | 0.007 | 0.0146^{b} | 0.007 |
| 3βHSD | A4/DHEA | 5.21 | 3.64 | 3.16 |
| 17HSD3 | Te/A4 | 3.50 | 3.63 | 4.22 |
| CYP19 | E1/A4 | 5.18 | 5.79 | 5.60 |
| CYP19 | E2/Te | 9.27 | 5.44 | 12.67^{b} |
| 17βHSD1 type 1 and 2 | E2/E1 | 6.21 | 3.80^{a} | 8.70 |

*Non-parametric test (Wilcoxon two-group test);
^{a}p < 0.05,
^{b}p < 0.01

The invention provides novel descriptions of steroid concentrations in FF from women diagnosed with PCOS and from regularly menstruating women, thereby providing means for determining the underlying causes in more detail. Simultaneous measurement of multiple steroids provides a better understanding of the underlying mechanisms and processes involved in the regulation of the menstrual cycle, ovulation and anovulation. In addition, the invention provides diagnostic and/or prognostic methods that allow for the tailoring and fine-tuning of IVF regimens to reach the goal of successful ovulation and pregnancy.

The invention provides a panel of laboratory tests that provide a diagnostic test for PCOS and related conditions or diseases relating to ovarian function, such as hyperandrogenism, reproductive abnormalities, infertility, menstrual disorders, anovulation, and can be useful for identification of the underlying deficiencies in ovarian function which are the cause of these and similar conditions. The invention also provides a diagnostic and/or prognostic test that may be used to refine stimulation regimens during fertility treatment, such as IVF, for selecting oocytes having a higher probability of achieving viable pregnancy, as well as for selecting oocytes which have low probability of achieving viable pregnancy, and, therefore, can be used for other purposes, such as production of embryonic stem cells for research or therapy. The invention further provides a method of analyzing the output or affect of potential drug candidates on ovarian function.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

All references, including publications, patents, and patent applications, cited herein, and contained in the following list, are hereby incorporated by reference to the same extent as if each reference were individually and specifically indicated to be incorporated by reference and were set forth in its entirety herein.
1. Speroff L, et al, (1999), Chapter 6: Regulation of the menstrual cycle. In: Mitchell C, editor. Clinical gynecologic endocrinology and infertility. 6th ed. Philadelphia, Pa.: Lippincott Williams & Wilkins; 202-246.
2. Brailly S, et al. (1981), Androgens and progestins in the human ovarian follicle: differences in the evolution of preovulatory, healthy nonovulatory, and atretic follicles J. Clin. Endocrinol. Metab 53:128-134.
3. Itskovitz J, et al. (1991), Relationship of follicular fluid prorenin to oocyte maturation, steroid levels, and outcome of in vitro fertilization J. Clin. Endocrinol. Metab 72:165-171,
4. Van Dessel H J H M, et al. (1996), Normal human follicle development: and evaluation of correlations with oestradiol, androstenedione and progesterone levels in individual follicles. Clin Endocrinol (Oxf) 44:191-198.
5. Franks S, (1989), Polycystic ovary syndrome: a changing perspective. Clin. Endocrinol (Oxf), 31(1):87-120.
6. Hillier S G, et al. (1980), Intraovarian sex steroid hormone interactions and the regulation of follicular maturation: aromatization of androgens by human granulosa cells in vitro. J Clin Endocrinol Metab, 50(4):640-647.
7. Hillier S G, et al. (1981), Control of preovulatory follicular estrogen biosynthesis in the human ovary. J Clin Endocrinol Metab, 52(5):847-856.
8. Lewicka S, et al. (2003), Cortisol and cortisone in human follicular fluid and serum and the outcome of IVF treatment. Hum Reprod. 18:1613-7.
9. Michael, A E, et al. (1999) Relationship between ovarian cortisol:cortisone ratios and the clinical outcome of in vitro fertilization and embryo transfer (IVF-ET). Clin Endocrinol 51:535-40.
10. Andersen C Y et al. (1999), Assessment of the follicular cortisol:cortisone ratio. Hum Reprod. 14:1562-8.
11. Basuray R, et al. (1988) High progesterone/estradiol ratio in follicular fluid at oocyte aspiration for in vitro fertilization as a predictor of possible pregnancy. Fertil Steril. 49:1007-11.
12. Franchimont P, et al. (1989) Correlation between follicular fluid content and the result of in vitro fertilization and embryo transfer. I. Sex steroids. Fertil Steril. 52:1006-11
13. Andersen C Y, et al. (1993) Characteristics of human follicular fluid associated with successful conception after in vitro fertilization. J Clin Endocrinol Metab. 77:1227-34.
14. De Sutter P, et al. (1991). Correlation between follicular fluid steroid analysis and maturity and cytogenetic analysis of human oocytes that remained unfertilized after in vitro fertilization. Fertil Steril 55:958-963.
15. Bergh C, Carlström K, Selleskog U, Hillensjö, T (1996), Effect of growth hormone on follicular fluid androgen levels in patients treated with gonadotropins before in vitro fertilization. Eur J Endocrinol 134:190-196.
16. Smitz J, et al. p (2007), Endocrine profile in serum and follicular fluid differs after ovarian stimulation with HP-hMG or recombinant FSH in IVF patients. Hum Reprod 22:676-687.
17. Taieb J, et al. (2002), Limitations of steroid determination by direct immunoassay, Clin Chem 48:583-585.
18. Dehennin L. (1990), Estrogens, androgens, and progestins in follicular fluid from preovulatory follicles: identification and quantification by gas chromatography/mass spectrometry associated with stable isotope dilution. Steroids. 55:181.4.
19. Dehennin L, et al, (1987), Androgen and 19-norsteroid profiles in human preovulatory follicles from stimulated cycles: an isotope dilution-mass spectrometric study. J Steroid Biochem 26:399-405.
20. Kushnir M M, et al. (2004), Cortisol and cortisone analysis in serum and plasma by atmospheric pressure photoionization tandem mass spectrometry. Clin Biochem, 37(5):357-362.
21. Kushnir M M, et al., (2006), Development and performance evaluation of a tandem mass spectrometry assay for 4 adrenal steroids. Clin Chem, 52(8):1559-1567.
22. Kushnir M M, et al. (2006), Performance characteristics of a novel tandem mass spectrometry assay for serum testosterone. Clin Chem, 52(1):120-128.
23. Kushnir M M, et al. (2008), A tandem mass spectrometry assay for estrone and estradiol in serum of postmenopausal women, men and children. American J Clin Path, 129:530-539.
24. M M Kushnir, et al. (2007). High sensitivity tandem mass spectrometry test for serum estrogens. Clin Chem 53:A183.
25. Kushnir M M, et al. (2008), Steroid Profiles in Ovarian Follicular Fluid from Regularly Menstruating Women and from Women after Ovarian Stimulation, Clin. Chem 55:519-526.
26. Kushnir M M, et al. (2005), Assessing analytical specificity in quantitative analysis using tandem mass spectrometry. Clin Biochem, 38(4):319-327.
27. Klein N A, et al. Soules M R (1996), Ovarian Follicular Development and the Follicular Fluid Hormones and Growth Factors in Normal Women of Advanced Reproductive Age. J Clin Endocrinol Metab 81:1946-1951.
28. Adams J, et al. (1986), Prevalence of polycystic ovaries in women with anovulation and idiopathic hirsutism. Br Med J (Clin Res Ed) 293(6543):355-359.
29. Ferriman D, and Gallwey G, (1961), Clinical assessment of body hair growth in women. J Clin Endoc Metab 21:1440-1447.

What is claimed is:

1. A method of diagnosing an endocrine-related condition relating to ovarian function, the method comprising:
obtaining a sample of ovarian follicular fluid from a subject;
analyzing the sample for at least one of plurality of steroids;
determining the concentration of at least one of said plurality of steroids in the sample;
evaluating the concentration of at least one of the plurality of steroids, or a ratio of concentrations of said steroids in comparison to at least one reference value characteristic of a given endocrine-related condition or diagnostic outcome; and
determining whether the subject is likely or not likely to have a given endocrine-related condition.

2. The method according to claim 1, where the endocrine condition is polycystic ovary syndrome.

3. The method according to claim 1 wherein the sample is analyzed using liquid chromatography followed by mass spectrometry.

4. The method according to claim 3 wherein the mass spectrometry is tandem mass spectrometry.

5. The method according to claim 1, wherein determining and evaluating the concentration of at least one of a plurality of steroids comprises analyzing, determining and evaluating the concentration of steroids selected from the group consisting of estrone, estradiol, estriol, DHEA, 17 hydroxypregnenolone, androstenedione, testosterone, androstanedione, 17 hydroxyprogesterone, pregnenolone, hydroxypregnenolone, allopregnanolone, progesterone, 11 deoxycortisol, cortisol, cortisone, and combinations and ratios thereof.

6. The method according to claim 1 further comprising identifying at least one biomarker from the plurality of steroids from said sample and comparing the concentration of said at least one biomarker with the values of the same biomarker in individuals not having the endocrine-related condition, wherein the higher or lower concentration of said at least one biomarker is an indication of said subject being afflicted with the endocrine-related condition.

7. The method according to claim 6, wherein said at least one biomarker is selected from the group consisting of 17 hydroxypregnenolone, androstenedione, total glucocorticoids, 11 deoxycortisol, cortisol, cortisone, androstanedione, estrone, estradiol, estriol, total androgens and ratios of 17 hydroxypregnenolone/pregnenolone, total estrogens/total androgens, estradiol/testosterone, DHEA/17 hydroxypregnenolone and combinations thereof.

8. The method according to claim 7, wherein elevated concentrations of at least one of said biomarkers from the group consisting of 17 hydroxypregnenolone, androstenedione, total glucocorticoids, 11 deoxycortisol, cortisol, cortisone, androstanedione, total androgens and ratios of 17 hydroxypregnenolone/pregnenolone indicates that the subject is likely to be afflicted with polycystic ovary syndrome.

9. The method according to claim 7, wherein reduced concentrations of at least one of said biomarkers from the group consisting of estrone, estradiol, estriol, total estrogens or ratios of total estrogens/total androgens, estradiol/testosterone, DHEA/17 hydroxypregnenolone indicates that the subject is likely to be afflicted with polycystic ovary syndrome.

10. The method according to claim 1 further comprising analyzing the concentration of selected steroids in said plurality of steroids and ratios of concentrations of steroids to detect deficiencies in the activity of enzymes in the pathway of biosynthesis of steroids in ovarian follicles as a means of diagnosing and guiding a treatment of patients.

11. A method of providing a prognosis for in vitro fertilization treatment or outcome, the method comprising:
obtaining a sample of ovarian follicular fluid from a subject;
analyzing a plurality of steroids from the sample;
determining the concentration of at least one steroid from said plurality of steroids;
evaluating the concentration of at least one steroid from said plurality of steroids or the ratio of concentrations of at least two steroids of said plurality of steroids in comparison with one or more reference values characteristic of a given outcome; and
determining the prognosis of a selected outcome based on said evaluation.

12. The method according to claim 11 wherein determining the prognosis of a selected outcome comprises determining that an oocyte is more likely to result in a successful pregnancy.

13. The method according to claim 11 wherein said selected outcome is the prognosis of likely viability of oocytes for a successful in vitro fertilization outcome.

14. The method according to claim 11 wherein said selected outcome is the prognosis for the likely non-viability of an oocyte for a successful in vitro fertilization outcome.

15. The method according to claim 14 wherein said prognosis for the likely non-viability of an oocyte for successful in vitro fertilization outcome further comprises determination of the suitability of said oocyte for use in subsequent embryonic stem cell-related procedures.

16. The method according to claim 11 further comprising identifying at least one biomarker in said plurality of steroids that have been analyzed and comparing the concentration of said at least one biomarker from said sample with the same biomarker from samples associated with other subjects who did not achieve a viable pregnancy and with the concentration of the same biomarker from samples of subjects who achieved a viable pregnancy, wherein the higher or lower concentration of said at least one biomarker in said sample is an indication of a selected outcome.

17. The method according to claim 16 wherein said at least one biomarker is selected from the group consisting of 17 hydroxyprogesterone, progesterone, 11 deoxycortisol, estriol, estrone, estradiol, pregnenolone, andostenedione, cortisol, cortisone, DHEA, 17 hydroxypregnenolone, hydroxyprogesterone, total pregnenolones, total estrogens, total androgens, total glucocorticoids and ratios of 17 hydroxypregnenolone/pregnenolone, 17 hydroxyprogesterone/progesterone, estradiol/estrone, estriol/estradiol, estriol/estrone, pregnenolone/allopregnanolone, androstenedione/11 deoxycortisol and combinations thereof.

18. The method according to claim 17, wherein increased concentration of at least one of the steroids of the group comprising 17 hydroxyprogesterone, hydroxyprogesterone, 11 deoxycortisol, estrone, estradiol, pregnenolone, 17 hydroxypregnenolone, total pregnenolones and ratios of 17 hydroxypregnenolone/pregnenolone, estradiol/estrone, estriol/estrone, pregnenolone/allopregnalone or combinations thereof, in said sample to predict the decreased likelihood of a successful in vitro fertilization outcome.

19. The method according to claim 17 wherein reduced concentration of at least one the steroids of the group comprising 17 hydroxyprogesterone, hydroxyprogesterone, 11 deoxycortisol, estriol, estrone, estradiol, andostenedione, cortisone, DHEA, total estrogens, total androgens, total glucocorticoids, total pregnenolones and ratios of estradiol/estrone, estriol/estrone, pregnenolone/allopregnnolone, androstenedione/11 deoxycortisol and combinations thereof, in said sample is predictive of decreased likelihood of a successful in vitro fertilization outcome.

20. The method according to claim 11 further comprising analyzing the concentration of selected steroids and precursors of selected steroids in said plurality of steroids to detect deficiencies in the activity of enzymes in the pathway of biosynthesis of steroids in ovarian follicles as a means of diagnosing and predicting the successful or unsuccessful outcome of in vitro fertilization or for guiding a treatment.

21. The method according to claim 20 further comprising determining the ratios of concentrations of steroid products and precursors of the pathway as representational of enzyme activities in ovarian follicles and using the ratios as a means of diagnosing and predicting the probability of a selected outcome of in vitro fertilization or for guiding a treatment.

22. A method of determining the suitability of oocytes for a selected use or procedure, comprising:
obtaining a sample of ovarian follicular fluid from a subject;
analyzing said sample for a plurality of steroids;
determining the concentration of at least one steroid or biomarker from said plurality of steroids;
comparing the concentration of said at least one steroid or biomarker from said sample with the concentration of the same at least one steroid or biomarker in the ovarian follicular fluid sample corresponding to oocytes which resulted in viable pregnancies; and
determining from said comparison a selected suitable use for the oocyte of the follicle from which said sample of ovarian fluid was taken.

23. The method according to claim 22 wherein the selected suitable use is in vitro fertilization.

24. The method according to claim 22 wherein the selected suitable use is embryonic stem cell-related procedures.
